(19) United States
(12) Patent Application Publication
(10) Pub. No.: US 2011/0076697 A1
(43) Pub. Date: Mar. 31, 2011

(54) LATERAL-FLOW IMMUNO-CHROMATOGRAPHIC ASSAY DEVICES

(75) Inventors: Igor Ruvinsky, Ramat Gan (IL); Ella Sklan, Jerusalem (IL)

(73) Assignee: INNOVATIVE LABORATORY TECHNOLOGIES, INC., New York, NY (US)

(21) Appl. No.: 12/768,706

(22) Filed: Apr. 27, 2010

Related U.S. Application Data
(60) Provisional application No. 61/173,596, filed on Apr. 28, 2009.
(60) Provisional application No. 61/268,844, filed on Jun. 17, 2009.

(51) Int. Cl. G01N 33/53; C12M 1/34

(52) U.S. Cl. 435/7.25; 435/287.2

(57) ABSTRACT

Device including migration membrane, conjugate pad on migration membrane, plasma separation membrane on conjugate pad, pre-filter on plasma separation membrane. Migration membrane has test line configured for loading one or plurality of capture antibodies having specific binding affinity for assay target. Migration membrane configured for allowing lateral flow of blood plasma or serum across migration membrane to test line. Conjugate pad configured for loading one or plurality of detection antibodies having specific binding affinity for assay target. Plasma separation membrane configured for allowing passage of blood plasma or serum and trapping erythrocytes. Pre-filter configured for loading assay sample including erythrocytes and either or both blood plasma and blood serum. Pre-filter configured for allowing passage of blood plasma or serum through pre-filter and causing lateral flow of blood plasma or serum within pre-filter. Method includes providing device and carrying out diagnostic assay cycle.

### BACKGROUND

1. Field of the Invention

This invention generally relates to lateral-flow immuno-chromatographic assay devices.

2. Related Art

Various types of lateral-flow immuno-chromatographic assay devices have been developed for carrying out diagnostic assays on blood samples. Lateral-flow immuno-chromatographic assay devices typically include an arrangement of layers of materials in a housing having openings for sample introduction and for reading of assay results.

There is a continuing need for lateral-flow immuno-chromatographic assay devices having structures and modes of utilization facilitating improved assay performance capabilities.

### SUMMARY

In an example of an implementation, a device is provided that includes a migration membrane, a conjugate pad on the migration membrane, a plasma separation membrane on the conjugate pad, and a pre-filter on the plasma separation membrane. The migration membrane has a test line configured for loading onto the test line of one or a plurality of capture antibodies having specific binding affinity for an assay target. The migration membrane is configured for allowing lateral flow of blood plasma or serum across the migration membrane to the test line. The conjugate pad is configured for loading onto the conjugate pad of one or a plurality of detection antibodies having specific binding affinity for an assay target. The plasma separation membrane is configured for allowing passage of blood plasma or serum through the plasma separation membrane and for trapping erythrocytes. The pre-filter is configured for loading of an assay sample including erythrocytes and either or both of blood plasma and blood serum onto the pre-filter. Further, the pre-filter is configured for allowing passage of blood plasma or serum through the pre-filter, and configured for causing lateral flow of blood plasma or serum within the pre-filter.

As another example of an implementation, a method is provided. The method includes providing a lateral-flow immuno-chromatographic assay device including a migration membrane, a conjugate pad being on the migration membrane, a plasma separation membrane being on the conjugate pad, and a pre-filter being on the plasma separation membrane. In the lateral-flow immuno-chromatographic assay device, the conjugate pad is loaded with one or a plurality of detection antibodies having specific binding affinity for an assay target; and the migration membrane has a test line loaded with one or a plurality of capture antibodies having specific binding affinity for the assay target. The method further includes carrying out a diagnostic assay cycle. In the diagnostic assay cycle, an assay sample including erythrocytes and either or both of blood plasma and blood serum is loaded onto the pre-filter. Also in the cycle, blood plasma or serum is caused to laterally flow within the pre-filter and allowed to pass through the pre-filter to the plasma separation membrane. The diagnostic assay cycle further includes causing erythrocytes to be trapped in the plasma separation membrane; and allowing blood plasma or serum to pass through the plasma separation membrane to the conjugate pad. Additionally, the cycle includes allowing blood plasma or serum to pass through the conjugate pad onto the migration membrane and allowing blood plasma or serum to laterally flow across the migration membrane to the test line.

Other devices, methods, features and advantages of the invention will be or will become apparent to one with skill in the art upon examination of the following figures and detailed description. It is intended that all such additional devices, methods, features and advantages be included within this description, be within the scope of the invention, and be protected by the accompanying claims.

### BRIEF DESCRIPTION OF THE FIGURES

The invention can be better understood with reference to the following figures. The components in the figures are not necessarily to scale, emphasis instead being placed upon illustrating the principles of the invention. Moreover, in the figures, like reference numerals designate corresponding parts throughout the different views.

FIG. 1 is an exploded perspective view illustrating an example of an implementation of a lateral-flow immuno-chromatographic assay device.

FIG. 2 is a cross-sectional view, taken along line A-A, of the lateral-flow immuno-chromatographic assay device shown in FIG. 1.

FIG. 3 is a flow diagram illustrating an example of an implementation of a method.

FIG. 4 is a photograph showing the lateral-flow immuno-chromatographic assay devices utilized to carry out Examples A, B and C.

FIG. 5 is a photograph showing the lateral-flow immuno-chromatographic assay devices utilized to carry out Examples D, E, F and G.

FIG. 6 is a photograph showing the lateral-flow immuno-chromatographic assay devices utilized to carry out Examples H, I, J and K.

FIG. 7 is a photograph showing the lateral-flow immuno-chromatographic assay devices utilized to carry out Examples L, M, N and O.

FIG. 8 is a photograph showing the lateral-flow immuno-chromatographic assay devices utilized to carry out Examples P, Q, R and S.

FIG. 9 is a photograph showing the lateral-flow immuno-chromatographic assay devices utilized to carry out Examples T, U, V and W.

FIG. 10 is a photograph showing the lateral-flow immuno-chromatographic assay devices utilized to carry out Examples X, Y, Z and AA.

FIG. 11 is a photograph showing the lateral-flow immuno-chromatographic assay devices utilized to carry out Examples AB, AC, AD and AE.

FIG. 12 is a photograph showing the lateral-flow immuno-chromatographic assay devices utilized to carry out Examples AF, AG, AH and AI.

FIG. 13 is a photograph showing the lateral-flow immuno-chromatographic assay devices utilized to carry out Examples AJ and AK.

FIG. 14 is a photograph showing the lateral-flow immuno-chromatographic assay devices utilized to carry out Examples AL and AM.

FIG. 15 is a photograph showing the lateral-flow immuno-chromatographic assay devices utilized to carry out Examples AN and AO.

FIG. 16 is a photograph showing the lateral-flow immuno-chromatographic assay device utilized to carry out Example AP.

FIG. 17 is a photograph showing the lateral-flow immuno-chromatographic assay devices utilized to carry out Examples AQ and AR.

### DETAILED DESCRIPTION

A lateral-flow immuno-chromatographic assay device may function, for example, by carrying a diagnostic assay sample to a conjugate pad loaded with one or a plurality of detection antibodies having specific binding affinity for an assay target. The diagnostic assay sample may then be laterally carried across a migration membrane to a test line loaded with one or a plurality of capture antibodies also having specific binding affinity for the assay target. If a sufficient concentration of the assay target was present in the diagnostic assay sample, then a detectable quantity of the detection antibodies may specifically bind with the assay target at the conjugate pad and may then laterally flow along with the assay sample to the test line. The capture antibodies at the test line may then also specifically bind there with the assay target, generating a visible mark constituting a positive test result of the diagnostic assay. For example, the detection antibodies may be tagged with a colored marker such as colloidal gold. Where the assay sample carries a sufficient quantity of the detection antibodies to the test line and then a sufficient quantity of the capture antibodies bind the assay target carrying the bound detection antibodies at the test line, a visibly colored mark may be formed. Where the detection antibody marker is colloidal gold, for example, a mark having a reddish, pinkish, or brownish hue may be formed.

The diagnostic assay sample may include red blood cells, also being referred to herein as “erythrocytes.” For example, the diagnostic assay sample may include whole blood, or may otherwise include erythrocytes and either or both of blood plasma and blood serum. Blood plasma or blood serum may, for example, be separated from erythrocytes before carrying out a lateral-flow immuno-chromatographic diagnostic assay to reduce or substantially eliminate the reddish color caused by hemoglobin. However, if erythrocytes in an assay sample become ruptured before or during performance of a lateral-flow immuno-chromatographic assay, hemoglobin released from the ruptured erythrocytes may stain the blood plasma or serum. If blood plasma or serum stained by hemoglobin reaches the test line in a lateral-flow immuno-chromatographic assay, then a visibly colored mark may be formed by the hemoglobin at the test line, generating a false positive assay result. These false positive results may be avoided by removing erythrocytes from an assay sample before carrying out a lateral-flow immuno-chromatographic diagnostic assay. For example, whole blood may be centrifuged to remove erythrocytes, so that the assay sample to be tested is in the form of blood plasma. Further, blood coagulation factors may also be removed, so that the assay sample tested is blood serum. However, in some cases, utilization of an assay sample including erythrocytes as well as either or both blood plasma and blood serum may be needed. For example, a medical professional may need to carry out a lateral-flow immuno-chromatographic diagnostic assay where equipment such as a centrifuge for removal of erythrocytes from whole blood is not available, or where rapid test performance is needed, such that waiting for preparation of a blood plasma or serum sample becomes an unacceptable delay. For example, a medical professional may need to carry out a lateral-flow immuno-chromatographic diagnostic assay at a location away from clinical facilities, or where a patient suffers from a critical, life-threatening condition.

As another example, a layman lacking both the skill and equipment needed to prepare a blood plasma or serum sample may need to himself carry out a lateral-flow immuno-chromatographic assay using whole blood. Further, such a layman may also lack both the equipment and skill needed to collect venous blood from a patient. Moreover, a layman may be reluctant to collect more than a single drop of the patient's capillary blood by a fingertip lance, especially if the layman needs to collect and then perform a diagnostic assay on his own blood. Additionally, such a layman may need to self-administer a lateral-flow immuno-chromatographic assay while suffering from a life-threatening condition at a location distant from professional medical personnel.

Hence, a lateral-flow immuno-chromatographic assay device capable of utilization with a small assay sample including erythrocytes and either or both of blood plasma and blood serum, such as a minimal sample of whole blood, may be useful in a variety of circumstances. Such a lateral-flow immuno-chromatographic assay device may, for example, need to effectively separate erythrocytes from blood plasma or serum so that false positive results due to ruptured erythrocytes are avoided. Further, for example, such a lateral-flow immuno-chromatographic assay device may need to effectively deliver as much of a small sample of blood plasma or serum as possible to the test line, so that a qualitative assay test result may be generated utilizing a small assay sample, such as a single drop of whole blood.

A lateral-flow immuno-chromatographic assay device may include a plasma separation membrane configured for allowing passage of blood plasma or serum through the plasma separation membrane and for trapping erythrocytes. In so trapping erythrocytes, the plasma separation membrane may become partially blocked and then impede the flow of blood plasma or serum through the plasma separation membrane toward the test line, detracting from a capability of the lateral-flow immuno-chromatographic assay device for utilization with an assay sample having a minimal volume, such as a single drop of blood. Hence, lateral-flow immuno-chromatographic assay devices are needed that may be capable of utilization with an assay sample of minimal volume, such as a single drop, including erythrocytes and either or both of blood plasma and blood serum. Such lateral-flow immuno-chromatographic assay devices, and methods for carrying out lateral-flow immuno-chromatographic assays, are provided herein.

A lateral-flow immuno-chromatographic assay device is provided herein that includes a migration membrane, a conjugate pad being on the migration membrane, a plasma separation membrane being on the conjugate pad, and a pre-filter being on the plasma separation membrane. The migration membrane has a test line configured for loading onto the test line of one or a plurality of capture antibodies having specific binding affinity for an assay target. The migration membrane is configured for allowing lateral flow of blood plasma or serum across the migration membrane to the test line. The conjugate pad is configured for loading onto the conjugate pad of one or a plurality of detection antibodies having specific binding affinity for an assay target. The plasma separation membrane is configured for allowing passage of blood plasma or serum through the plasma separation membrane and for trapping erythrocytes. The pre-filter is configured for loading of an assay sample including erythrocytes and either or both of blood plasma and blood serum onto the pre-filter. The pre-filter is also configured for allowing passage of blood plasma or serum through the pre-filter. Further, the pre-filter is configured for causing lateral flow of blood plasma or serum within the pre-filter. The pre-filter may additionally be configured for causing selective passage of blood plasma or serum through the pre-filter and for trapping erythrocytes. The device may be configured for being capable of utilizing a single drop of whole blood, such as hanging drop for example, as the assay sample.

In examples, the one or plurality of detection antibodies and the one or plurality of capture antibodies may have specific binding affinity for a cardiac Troponin-I epitope. The plurality of detection antibodies may include cardiac Troponin-I antibody clone 19C7 together with either or both of cardiac Troponin-I antibody clones 4C2 and M155. The plurality of capture antibodies may include both cardiac Troponin-I antibody clones MF4 and 16A11. As another example, the lateral-flow immuno-chromatographic assay device may include first and second detection antibodies and third and fourth capture antibodies, each of the first, second, third and fourth antibodies having specific binding affinity for substantially different cardiac Troponin-I epitopes. Throughout this specification, the term “substantially different” as applied to two epitopes of an assay target means that the two epitopes are sufficiently different to allow two antibodies to bind the target simultaneously through binding to the two epitopes.

The following conventions apply regarding terminology utilized throughout this specification. A “layer” of a material is any component of a lateral-flow immuno-chromatographic assay device that is bonded or attached to, formed or deposited on, or otherwise provided on any other layer or on or in the housing of the lateral-flow immuno-chromatographic assay device. A layer may include, as examples, a mat, surface, film, foil, region, body, or substrate. When one layer or material is referred to as being “on”, “over”, or “loaded onto” another layer or housing, then all or a portion of the layer or material may be directly on and in contact with all or a portion of the other layer or housing, or alternatively, intervening layers may also be present such that all or portions of the one layer and of another layer that is “on” or “over” the one layer or housing are not mutually in direct contact. When a layer is stated as being “directly on” another layer or the housing, then no intervening layer is present unless otherwise indicated. When a layer is stated as being “between” two other layers, then one or more additional intervening layers may also be present between the two other layers. When one layer is referred to as being “on” (or “over”) another layer, then the one layer may cover the entire surface of the other layer, or may cover only a portion of the other layer. When a material is stated as being “loaded onto” a layer or a surface of a layer, the material may remain on a surface of the layer, or may also penetrate through the surface into the layer, or may penetrate into and pass through the layer. Terms such as “formed on”, “disposed on”, “loaded onto” or “deposited on” are not intended to introduce any limitations relating to specific methods for fabricating a layer except as otherwise designated.

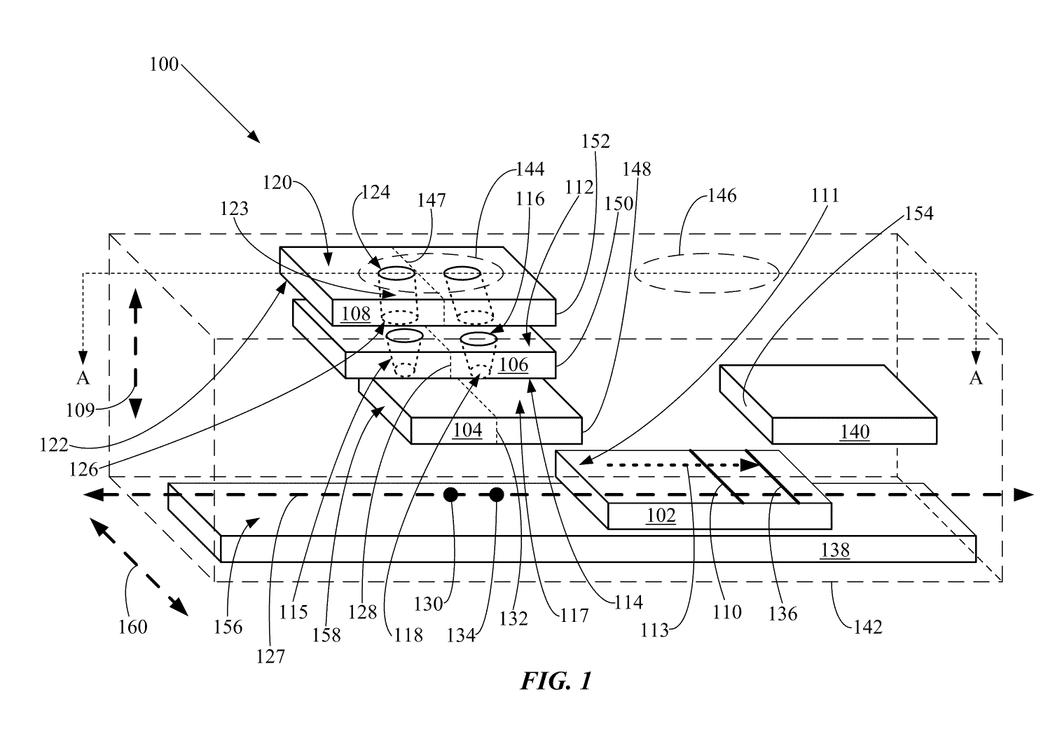

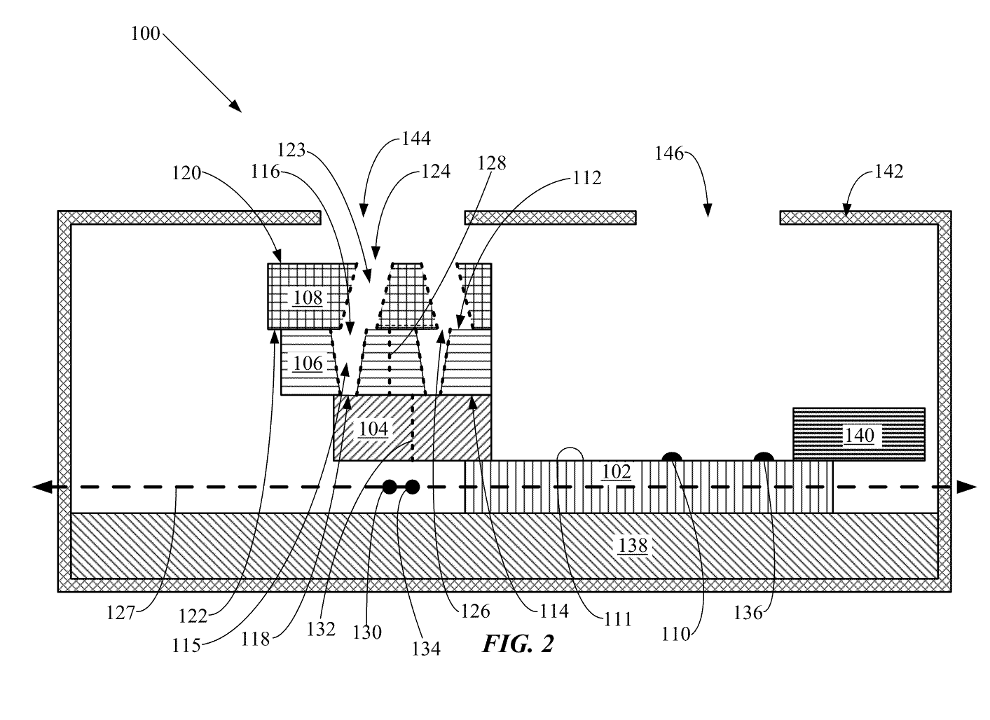

FIG. 1 is an exploded perspective view illustrating an example of an implementation of a lateral-flow immuno-chromatographic assay device 100. FIG. 2 is a cross-sectional view, taken along line A-A, of the lateral-flow immuno-chromatographic assay device 100 shown in FIG. 1. The lateral-flow immuno-chromatographic assay device 100 includes a migration membrane 102, a conjugate pad 104 being on the migration membrane 102, a plasma separation membrane 106 being on the conjugate pad 104, and a pre-filter 108 being on the plasma separation membrane 106. These components of the lateral-flow immuno-chromatographic assay device 100 are exploded in FIG. 1 in the directions of an arrow 109. The migration membrane 102 has a test line 110 configured for loading onto the test line 110 of one or a plurality of capture antibodies (not shown) having specific binding affinity for an assay target (not shown). So configuring the test line 110 may include forming the migration membrane 102 with a surface 111 selected as suitable for loading and binding the one or plurality of capture antibodies onto the surface 111 at the test line 110. The migration membrane 102 is also configured for allowing lateral flow of blood plasma or serum (not shown) across the migration membrane 102 to the test line 110. So configuring the migration membrane 102 may include arranging the conjugate pad 104, the migration membrane 102, and the test line 110 to form a pathway in the direction of an arrow 113 suitable to allow lateral flow of blood plasma or serum across the migration membrane 102 to the test line 110.

Throughout this specification, the term “blood plasma” means the components of whole blood from which the solid cellular components, including erythrocytes, leukocytes and thrombocytes, have been removed. Throughout this specification, the term “blood serum” means the components of whole blood from which the coagulants and the solid cellular components have been removed. Throughout this specification, all references to “blood plasma” are deemed to designate, except where expressly stated or clear from the context otherwise: “blood plasma” and “blood serum” together in a mixture, as well as either “blood plasma” alone or “blood serum” alone. Throughout this specification, all references to “blood plasma or serum” are deemed to collectively designate and include, except where expressly stated otherwise: “blood plasma”, “blood serum”, and “blood plasma and blood serum”.

Throughout this specification, the term “lateral flow” as applied to flow of blood plasma or serum across the migration membrane 102 means that the lateral-flow immuno-chromatographic assay device 100 is configured to allow flow of blood plasma or serum from the conjugate pad 104 to the test line 110. However, the orientation of the lateral-flow immuno-chromatographic assay device 100 shown in FIGS. 1-2 is for purposes of illustration and does not indicate a horizontal positioning or any other specific positioning of the lateral-flow immuno-chromatographic assay device 100 relative to gravity during utilization or otherwise.

The conjugate pad 104 is on the migration membrane 102, being configured for loading onto the conjugate pad 104 of one or a plurality of detection antibodies (not shown) having specific binding affinity for an assay target. So configuring the conjugate pad 104 may include forming the conjugate pad 104 with a surface 117 selected as suitable for loading the one or plurality of detection antibodies onto the surface 117. The one or plurality of detection antibodies may, for example, penetrate into the surface 117 and soak into the conjugate pad 104. The detection antibodies may include a visibly colored tagging agent, such as colloidal gold particles or blue latex microspheres, as examples. Colloidal gold particles having an average diameter of about 40 nanometers (nm) that may be utilized, and services of tagging antibodies with such particles, are commercially available from Arista Biologicals, having a business address at 1101 Hamilton Street, Allentown, Pa. 18101 USA. The plasma separation membrane 106 is configured for allowing passage of blood plasma or serum through the plasma separation membrane 106 and for trapping erythrocytes (not shown). The plasma separation membrane 106 may further be configured for causing selective passage of blood plasma or serum through the plasma separation membrane 106.

The pre-filter 108 is configured for loading of an assay sample (not shown) including erythrocytes and either or both of blood plasma and blood serum onto the pre-filter 108. As an example, the assay sample may include whole blood. In further examples, the assay sample may include whole blood together with either or both of blood plasma and blood serum. In additional examples, the assay sample may include either or both of blood plasma and blood serum, while not including blood cells. Although the pre-filter 108 is configured for loading of an assay sample that includes erythrocytes, it is understood throughout this specification that the lateral-flow immuno-chromatographic assay devices that are disclosed herein may be utilized for carrying out an immuno-chromatographic assay on an assay sample that does not include erythrocytes but that includes either or both of blood plasma and blood serum. For example, the assay sample to be utilized may be a sample of blood plasma or of blood serum.

Configuring the pre-filter 108 for loading of an assay sample including erythrocytes and either or both of blood plasma and blood serum onto the pre-filter may include, for example, forming the pre-filter 108 with a first surface 120 selected as suitable for loading such an assay sample onto the surface 120. The pre-filter 108 is also configured for allowing passage of blood plasma or serum through the pre-filter 108 and configured for causing lateral flow of blood plasma or serum within the pre-filter 108. Configuring the pre-filter 108 for allowing such passage of blood plasma or serum and for causing such lateral flow may include, for example, selecting a pre-filter 108 having a fibrous structure, or having a structure including pores forming pathways communicating between the first surface 120 and a second surface 122.

In an example, the plasma separation membrane 106 may have a first surface 112 facing toward the pre-filter 108 and a second surface 114 facing toward the conjugate pad 104. Configuring the plasma separation membrane 106 for allowing passage of blood plasma or serum through the plasma separation membrane 106 and for trapping erythrocytes may include providing the plasma separation membrane 106 with a fibrous structure, or having a structure including pores forming pathways communicating between the first surface 112 and the second surface 114. In another example, the plasma separation membrane 106 may include a plurality of passageways 115 each communicating with both of the first and second surfaces 112, 114, and wherein a plurality of the passageways 115 each has a first opening 116 at the first surface 112 and a second opening 118 at the second surface 114, the second opening 118 being smaller than the first opening 116. Each of a plurality of the passageways 115 may have a frustoconical shape, the plurality of passageways 115 being laterally spaced apart from each other within the plasma separation membrane 106. The frustoconical shape of each of the plurality of passageways 115 may be configured for trapping and immobilizing erythrocytes.

The pre-filter 108 may, for example, have a random structure configured for allowing omni-directional passage of an assay sample, such as an assay sample including whole blood, or otherwise including erythrocytes and either or both of blood plasma and blood serum, or an assay sample including either or both of blood plasma and serum, through the pre-filter 108. The pre-filter 108 may have a random fibrous structure. The pre-filter 108 may be configured for causing selective passage of blood plasma or serum through the pre-filter 108 and for trapping erythrocytes. The pre-filter 108 may be configured for trapping at least a minimum proportion of the erythrocytes from an assay sample sufficient to significantly reduce a tendency of the plasma separation membrane 106 to become blocked by erythrocytes, thereby improving the plasma separation membrane's performance in trapping erythrocytes and in allowing blood plasma or serum to flow through the plasma separation membrane 106. For example, the pre-filter 108 may be configured for trapping at least about 10%, or at least about 30%, of a quantity of erythrocytes from an assay sample. The pre-filter 108 may be configured for causing substantial lateral flow of an assay sample, such as an assay sample including whole blood, or otherwise including erythrocytes and either or both of blood plasma and blood serum, or an assay sample including either or both of blood plasma and serum, within the pre-filter 108. Throughout this specification, the term “substantial lateral flow” as applied to flow of blood plasma or serum through the pre-filter 108 means that the blood plasma or serum exits from the pre-filter 108 through a portion of the second surface 122 having an area at least about 5% larger than an area on the first surface 120 of the pre-filter 108 through which the blood plasma or serum enters the pre-filter 108. Throughout this specification, the term “substantial lateral flow” as applied to flow of an assay sample including erythrocytes through the pre-filter 108 means that the assay sample exits from the pre-filter 108 through a portion of the second surface 122 having an area at least about 5% larger than an area on the first surface 120 of the pre-filter 108 through which the assay sample enters the pre-filter 108.

The pre-filter 108 may, for example, have an exposed first surface 120 and a second surface 122 facing toward the plasma separation membrane 106. The pre-filter 108 may have a structure including a plurality of passageways 123 having first openings 124 communicating with the first surface 120 and second openings 126 communicating with the second surface 122. The pre-filter 108 may be configured for causing an assay sample, such as an assay sample including whole blood, or otherwise including erythrocytes and either or both of blood plasma and blood serum, or an assay sample including either or both of blood plasma and serum, to flow out of a larger quantity of second openings 126 than a quantity of first openings 124 through which the assay sample enters the pre-filter 108. The pre-filter 108 may cooperate with the plasma separation membrane 106 to convey a greater portion of the assay sample, such as an assay sample including whole blood, or otherwise including erythrocytes and either or both of blood plasma and blood serum, to the conjugate pad 104 than the plasma separation membrane 106 would be capable of so conveying without the pre-filter 108. In this regard, the pre-filter 108 may cause the assay sample to laterally spread over an enlarged portion of the first surface 112 of the plasma separation membrane 106. The assay sample, such as an assay sample including whole blood, or otherwise including erythrocytes and either or both of blood plasma and blood serum, then passes into an enlarged portion of the plasma separation membrane 106, allowing blood plasma or serum to flow through the plasma separation membrane 106 and allowing erythrocytes to be trapped in a larger quantity of the passageways 115. As trapped erythrocytes are spread over a larger quantity of the passageways 115, blockage to flow of blood plasma or serum through the plasma separation membrane 106 is accordingly reduced. Further, the blood plasma or serum may then likewise flow onto an enlarged portion of the conjugate pad 104, allowing the detection antibodies to make contact with the blood plasma or serum over an enlarged area. As another example, the pre-filter 108 may have an asymmetric structure wherein an average spacing between the second openings 126 is larger than an average spacing between the first openings 124.

The pre-filter 108 and the plasma separation membrane 106 may, for example, be collectively configured for trapping at least about 90% of a quantity of erythrocytes from an assay sample. The lateral-flow immuno-chromatographic assay device 100 may be configured for conveying a substantial portion of the blood plasma or serum from an assay sample to the migration membrane 102. It is understood throughout this specification that the term “substantial portion” means that at least about 60% by volume of the blood plasma or serum from an assay sample is conveyed to the migration membrane 102. For example, the lateral-flow immuno-chromatographic assay device 100 may be configured for conveying between about 60% by volume and about 80% by volume of the blood plasma or serum from an assay sample to the migration membrane 102. The lateral-flow immuno-chromatographic assay device 100 may be configured for being capable of utilizing a single drop of whole blood, such as hanging drop for example, as the assay sample. A drop of blood may have a volume within a range of between about 20 microliters (μl) and about 65 μl. A hanging drop of blood may have a volume within a range of between about 60 μl and about 65 μl. The blood plasma or serum in a hanging drop of whole blood may have a volume within a range of between about 32 μl and about 42 μl. Where an assay sample includes either or both of blood serum and blood plasma, but does not include erythrocytes, an assay sample volume within that range, i.e. between about 30 μl and about 42 μl, may for example be utilized.

The lateral-flow immuno-chromatographic assay device 100 may be capable of carrying out a diagnostic assay on a small sample of whole blood, such as a single drop of whole blood, without a need to separate the blood plasma or serum from erythrocytes before loading the whole blood sample onto the lateral-flow immuno-chromatographic assay device 100. For example, centrifugation of whole blood before loading a drop of whole blood onto the pre-filter 108 may not be needed. Accordingly, the lateral-flow immuno-chromatographic assay device 100 may facilitate carrying out a diagnostic assay under circumstances where external cellular component-separating equipment for pre-treatment of an assay sample, such as a centrifuge, may be unavailable. For example, the lateral-flow immuno-chromatographic assay device 100 may be suitable for utilization “in the field”, away from any hospital or clinic, as a stand-alone portable diagnostic assay device. The capability of utilizing the lateral-flow immuno-chromatographic assay device 100 to carry out a diagnostic assay on a single drop of whole blood also facilitates utilization of the lateral-flow immuno-chromatographic assay device 100 by a layman, who may be able to self-draw the small needed sample of whole blood from a capillary by a routine finger prick with a lance, and to then himself carry out the diagnostic assay. Further for example, a layman who suspects that he or she has had or is having a heart attack may be able to successively self-administer a plurality of diagnostic assays utilizing a plurality of lateral-flow immuno-chromatographic assay devices 100, in order to monitor his or her own physical condition over a period of time as well as to provide ongoing status information to a remotely-located cardiologist.

The migration membrane 102 may have a longitudinal axis 127. The plasma separation membrane 106 may have a midpoint 128 tangentially located over a first point 130 along the longitudinal axis 127. The conjugate pad 104 may have a midpoint 132 tangentially located over a second point 134 along the longitudinal axis 127, and wherein the second point 134 is nearer to the test line 110 than is the first point 130. This relative orientation of the midpoints 130, 134 may serve to bias flow of blood plasma or serum toward the migration membrane 102 as the blood plasma or serum passes from the plasma separation membrane 106 to the conjugate pad 104 and then onto the migration membrane 102. The migration membrane 102 may also include a control line 136, on which a control test may be carried out. For example, antibodies selected as capable of binding the detection antibodies may be loaded at the control line 136 to verify proper assay functionality, including flow of the detection antibodies together with blood plasma or serum from the conjugate pad 104 to the test line 110. The lateral-flow immuno-chromatographic assay device 100 may further include a substrate 138, an absorption pad 140, and a housing 142. The substrate 138 may include an adhesive layer (not shown) for securing the migration membrane 102, conjugate pad 104, and absorption pad 140 on the substrate 138. The housing 142 may include an opening 144 for assay sample introduction and an opening 146 for reading of assay results. In an example, the opening 144 may be centered over a mid-point 147 of the pre-filter 108. During utilization of the lateral-flow immuno-chromatographic assay device 100 to carry out an assay, this orientation of the opening 144 may enable an assay sample to spread out in all directions over the first surface 120 of the pre-filter 108, to improve flow of blood plasma or serum.

When the lateral-flow immuno-chromatographic assay device 100 is utilized to carry out a diagnostic assay, an assay sample loaded onto the pre-filter 108 flows through the pre-filter 108 and then through the plasma separation membrane 106. Erythrocytes in the assay sample are trapped in the plasma separation membrane 106; and may also be trapped on or in the pre-filter 108. Leukocytes and thrombocytes, if present in the assay sample, may be trapped in the plasma separation membrane 106; and may also be trapped on or in the pre-filter 108. Blood plasma or serum then passes through the conjugate pad 104. The detection antibodies loaded onto the conjugate pad 104 then specifically bind with target antigen if present in the blood plasma or serum, and the blood plasma or serum then carries the bound detection antibodies laterally across the migration membrane 102 to the test line 110. The visibly colored agent, bound to detection antibodies specifically bound to the target antigen, is accordingly carried to the test line 110. The capture antibodies, which may be bound to the migration membrane 102 at the test line 110, then specifically bind with the target antigen if present in the blood plasma or serum, effectively binding the visibly colored agent to the test line 110. If the target antigen was present at a detectable concentration in the assay sample, then an accumulation of the visibly colored agent so bound may form a visible mark at the test line 110, being a positive qualitative assay result indicating the presence of the target antigen in the assay sample. Where the visibly colored agent is colloidal gold, the visible mark so formed at the test line 110 may have, as examples, a reddish, pinkish or brownish colored appearance.

As the diagnostic assay takes place, a visibly colored leading edge (not shown) formed by the visibly colored agent bound to detection antibodies specifically bound to the target antigen is carried to the test line 110. The migration membrane 102 may, for example, be configured for causing the leading edge of the visibly colored agent to be conveyed across the migration membrane 102 at a controlled speed within a range of between about 2.5 minutes per 3 centimeters (min/3 cm) and about 3.75 min/3 cm. The migration membrane 102 may be impregnated with a membrane blocking buffer at a concentration selected for causing the leading edge of the visibly colored agent to be conveyed at the controlled speed. The migration membrane 102 may have an average pore diameter selected for causing the leading edge of the visibly colored agent to be conveyed at the controlled speed. For example, a migration membrane 102 including pores having an average pore diameter of between about 1 micrometer (μm) and about 250 μm may be suitable for causing the leading edge of the visibly colored agent to be conveyed at the controlled speed. The selected migration membrane 102 may be electrically uncharged in furtherance of maintaining the controlled speed.

The physical dimensions of the lateral-flow immuno-chromatographic assay device 100 may be selected, for example, by establishing the flow rates of blood plasma or serum through the pre-filter 108, the plasma separation membrane 106, and the conjugate pad 104; and the flow rate of blood plasma or serum across the migration membrane 102 to the test line 110. These flow rates may then be utilized to define physical dimensions for the lateral-flow immuno-chromatographic assay device 100 such that a diagnostic assay may be carried out over and completed after a moderate period of time. It is understood throughout this specification that a “moderate” period of time is a time period of less than about 20 minutes. As an example, a moderate period of time may be a time period within a range of between about 7 minutes and about 20 minutes; or a time period within a range of between about 10 minutes and about 15 minutes. An excessively short assay completion time period may lead to inaccurate assay test results, for example because the blood plasma or serum may migrate too rapidly past the test line 110. In that case, the capture antibodies may not adequately bind with the assay target in the blood plasma or serum. An excessively long assay completion time period detracts from the usefulness of the lateral-flow immuno-chromatographic assay device 100, and may lead to false positive results. For example, such an excessively long assay completion time period may enable sufficient hemoglobin from ruptured erythrocytes in an assay sample to reach the test line 110 and accumulate there to generate a visible line to mimic the presence of detection antibody-bound target antigen. However, the lateral-flow immuno-chromatographic assay device 100 may also enable an accurate qualitative assay result to be observed over an extended time period, continuing after completion of a diagnostic assay, such extended time period being longer than a moderate period of time. For example, an accurate qualitative assay result may remain visible upon inspection of the lateral-flow immuno-chromatographic assay device 100 at any point over an extended time period within a range of between about 7 minutes and about 90 minutes following initiation of a diagnostic assay. Suitable physical dimensions for the lateral-flow immuno-chromatographic assay device 100 may further be selected according to a particular end-use application, such that a particular diagnostic assay may be effectively carried out. For example, the physical dimensions of the lateral-flow immuno-chromatographic assay device 100 may additionally take into consideration the flow rates of detection antibody-tagged target antigen through the conjugate pad 104 and across the migration membrane 102 to the test line 110.

As an example, the migration membrane 102 may have a length within a range of between about 22.0 millimeters (mm) and about 30.0 mm; or of about 25.0 mm. It is understood throughout this specification that all length dimensions of components in examples of the lateral-flow immuno-chromatographic assay device 100 are defined in directions of the arrow 127. It is further understood throughout this specification that all dimensions of components in examples of the lateral-flow immuno-chromatographic assay device 100, including lengths, widths, heights, relative proportions between dimensions, and any other dimensions, are examples for purposes of illustration; and that lateral-flow immuno-chromatographic assay devices 100 having other dimensions and proportions may be fabricated and utilized. The conjugate pad 104 may have a length within a range of between about 9.0 mm and about 12.0 mm; or of about 10.0 mm. The conjugate pad 104 may overlap with the migration membrane 102 over a length within a range of between about 0.5 mm and about 3.5 mm; or of about 2.0 mm. The test line 110 may be spaced apart from a trailing edge 148 of the conjugate pad 104 by a distance along the migration membrane 102 having a length within a range of between about 7.0 mm and about 12.0 mm; or of about 9.0 mm. The control line 136 may be spaced apart from the trailing edge 148 of the conjugate pad 104 by a distance along the migration membrane 102 having a length within a range of between about 12.0 mm and about 20.0 mm; or of about 17.0 mm. The plasma separation membrane 106 may have a length within a range of between about 11.8 mm and about 15.0 mm; or of about 13.0 mm. The pre-filter 108 may have a length within a range of between about 12.5 mm and about 15.0 mm; or of about 14.0 mm. A trailing edge 150 of the plasma separation membrane 106 and a trailing edge 152 of the pre-filter 108 may, for example, be mutually aligned together along the longitudinal axis 127 slightly farther away than the trailing edge 148 from the test line 110. The absorption pad 140 may have a length within a range of between about 19.0 mm and about 22.0 mm; or of about 20.0 mm. A leading edge 154 of the absorption pad 140 may overlap with the migration membrane 102 over a length within a range of between about 0.5 mm and about 2.0 mm; or of about 1.0 mm. The trailing edge 148 of the conjugate pad 104 may be spaced apart from the leading edge 154 of the absorption pad 140 by a distance along the migration membrane 102 having a length within a range of between about 21.0 mm and about 24.0 mm; or of about 22.0 mm. The substrate 138 may have a length within a range of between about 61.0 mm and about 63 mm; or of about 62.5 mm. The migration membrane 102 may be located along the length of the substrate 138 such that a portion of the substrate 138 defines a dead space 156. The dead space 156 may have a length within a range of between about 8.5 mm and about 11.5 mm; or of about 10.0 mm, extending away from a leading edge 158 of the conjugate pad 104. The dead space 156 may serve to orient the midpoint 147 of the pre-filter 108 in a position approximately centered along the longitudinal axis 127 relative to the opening 144 in the housing 142.

The lateral-flow immuno-chromatographic assay device 100 may have a width defined by directions of an arrow 160, and a height defined by directions of the arrow 109. The lateral-flow immuno-chromatographic assay device 100 may have a width 160 within a range of between about 8.0 mm and about 8.3 mm; or of about 8.2 mm. The lateral-flow immuno-chromatographic assay device 100 may have a height in directions of the arrow 109 within a range of between about 1.5 mm and about 1.8 mm; or of about 1.7 mm.

The pre-filter 108 may, for example, have a selected thickness within a range of between about 355 μm and about 508 μm. The plasma separation membrane 106 may, for example, have a selected thickness within a range of between about 310 μm and about 350 μm, or of about 330 μm. The conjugate pad 104 may, for example, have a selected thickness within a range of between about 355 μm and about 508 μm. The migration membrane 102 may, for example, have a selected thickness within a range of between about 165 μm and about 205 μm. The absorption pad 140 may, for example, have a selected thickness within a range of between about 304 μm and about 370 μm. The substrate 138 may, for example, have a selected thickness including an adhesive layer, within a range of between about 550 μm and about 650 μm

The pre-filter 108 is formed of a material having a structure suited for causing blood plasma or serum to laterally flow within the pre-filter 108, and also for allowing blood plasma or serum to pass through the pre-filter 108. The pre-filter 108 may have a random structure that is both porous to flow of blood plasma or serum and that causes such lateral flow to occur. The random structure may be a fibrous random structure. Such a material may also have a porosity suitably sized or a fiber density suitable for trapping some of the erythrocytes present in an assay sample. The pre-filter 108 may, for example, be formed of a cellulosic glass fiber material. In further examples, the pre-filter 108 may be formed of borosilicate glass fiber with a polyvinyl alcohol binder, having the grade designation “SMCON64” or “SMCON75”, both of which are commercially available from the Pall Corporation, having a business address at 2200 Northern Blvd., East Hills, N.Y. 11548 USA; www.pall.com. The entirety of the Pall Corporation's 3-page “Conjugate Pads” product data sheet including the SMCON64 and SMCON75 materials is hereby incorporated herein by reference.

The plasma separation membrane 106 is formed of a material having a structure suited for allowing blood plasma or serum to pass through the plasma separation membrane 106, and for trapping erythrocytes. For example, the plasma separation membrane 106 may be formed of an asymmetric membrane material having large pores 116 at the first surface 112 and smaller pores 118 at the second surface 114. As an example, the large pores 116 may have diameters of about 220 μm, and the small pores 118 may have diameters of about 2.5 μm. Erythrocytes may then be trapped in the large pores 116, while blood plasma or serum flows out of the plasma separation membrane 106 through the smaller pores 118. In an example, the plasma separation membrane 106 may be an asymmetric membrane material formed of a polysulfone and having the trade name “Vivid™ Plasma Separation Membrane” which is commercially available from the Pall Corporation. The entirety of the Pall Corporation's 6-page product data sheet for the Vivid™ Plasma Separation Membrane is hereby incorporated herein by reference.

The conjugate pad 104 is formed of a material having a structure suited for loading onto the conjugate pad 104 of one or a plurality of detection antibodies having specific binding affinity for an assay target. For example, the material may have a structure suited for allowing or causing the one or plurality of detection antibodies to penetrate into and soak the conjugate pad 104. The conjugate pad 104 may have a random structure that is porous to flow of blood plasma or serum. The random structure may be a fibrous random structure. Such a material may also have a porosity suitably sized or a fiber density suitable for trapping any remaining erythrocytes from an assay sample. The conjugate pad 104 may, for example, be formed of a cellulosic glass fiber material. In further examples, the conjugate pad 104 may be formed of borosilicate glass fibers with a polyvinyl alcohol binder, having the grade designation “SMCON64” or “SMCON75”, both of which are commercially available from the Pall Corporation. Further, for example, the conjugate pad 104 may be formed of a fibrous material having the grade designation “FUSION 5™”, commercially available from Whatman Inc., having a business address at Building 1, 800 Centennial Avenue, Piscataway, N.J. 08854 USA; www.whatman.com. The entirety of Whatman Inc.'s 2-page product data sheet for the Fusion 5™ material is hereby incorporated herein by reference. As another example, a binder- and surfactant-free hydrophilic fibrous material formed of hydroxylated polyester, having a basis weight of about 101 grams per square meter (g/m²), a hold-up volume of about 39 microliters per square centimeter (μl/cm²), a water wicking rate of about 44 seconds per 3 centimeters (44 sec/3 cm), and an absorption capacity of about 38 μl/cm², may be utilized.

The materials from which each of the pre-filter 108, the plasma separation membrane 106, and the conjugate pad 104 are formed, may take the form of sheets, flat discs, or webs, as examples. The materials may have a high water wicking rate to facilitate flow of blood plasma or serum into and through the pre-filter 108, the plasma separation membrane 106, and the conjugate pad 104. The materials from which each of the pre-filter 108, the plasma separation membrane 106, and the conjugate pad 104 are formed may further be selected to minimize binding of a target antigen to such material; and may be treated with a membrane blocking buffer to inhibit protein binding. The assay sample may be treated with an anti-coagulant such as a heparin salt, a citrate such as sodium citrate, or an ethylene diamine tetra-acetic acid (EDTA) salt before loading onto the pre-filter 108. Alternatively, the material from which the pre-filter 108 is formed may be treated with an anti-coagulant. In addition, the materials from which the pre-filter 108, the plasma separation membrane 106, and the conjugate pad 104 are formed may be treated with a surfactant suitable for causing such materials to be hydrophilic.

The migration membrane 102 is formed of a material having a structure suited to allow lateral flow of blood plasma or serum across the migration membrane 102 to the test line 110. As an example, the migration membrane 102 may be formed of a material selected as suitable to allow such lateral flow to occur over a selected period of time. Further, for example, the structure of the material utilized in forming the migration membrane 102 may actively cause the lateral flow of the blood plasma or serum to occur, such as by capillary action or wicking. The migration membrane 102 may be formed of a material having a structure including a generally uniform wicking rate, thickness, tensile strength, and protein binding level. The migration membrane 102 may have a generally uniform surface 111 with minimal scratches, dust and other irregularities. The migration membrane 102 may be a layer of material formed on a backing (not shown), where the backing has sufficient tensile strength to maintain the shape and integrity of the migration membrane 102 during fabrication and use of the lateral-flow immuno-chromatographic assay device 100. For example, the migration membrane 102 may include a nitrocellulose layer formed on a polyester backing. Further, the migration membrane 102 may be formed on such a backing without an adhesive, to avoid leaching of the adhesive into the blood plasma or serum when an assay is carried out. For example, the migration membrane 102 may be formed of a material selected as having a tensile strength of at least about 12 Newtons. Tensile strength may be measured on a sample of material for forming the migration membrane 102 having a width of 15 mm and a length of 1,000 mm, using the testing protocol in DIN 53 112, part 1; or utilizing ASTM D 828 “Standard Test Method for Tensile Properties of Paper and Paperboard Using Constant-Rate-of-Elongation Apparatus.” In an example, the migration membrane 102 may be a polyester-backed nitrocellulose membrane material having a wicking rate within a range of between about 150 sec/4 cm and about 225 sec/4 cm, a tensile strength of at least about 12 Newtons, and a protein binding (bovine serum albumin) rate within a range of between about 45 micrograms per square centimeter (μg/cm²) and about 59 μg/cm², sold under trade name “Vivid™ 170 Lateral Flow Nitrocellulose Membrane” by the Pall Corporation. The entirety of the Pall Corporation's 4-page product data sheet for the Vivid™ 170 Lateral Flow Nitrocellulose Membrane is hereby incorporated herein by reference. The migration membrane 102 may be impregnated with a membrane blocking buffer. The migration membrane 102 may be treated with a surfactant suitable for causing the migration membrane 102 to be hydrophilic.

The substrate 138 may be formed of a material having a structure suited for physically supporting the pre-filter 108, the plasma separation membrane 106, the conjugate pad 104, the migration membrane 102, and the absorption pad 140, and for maintaining such components of the lateral-flow immuno-chromatographic assay device 100 in position within the housing 142. For example, the substrate 138 may be formed of a rigid sheet material such as a high impact polystyrene sheet having a thickness of about 500 μm and including an adhesive layer. Such a material suitable for forming the substrate 138, having the grade designation “L-H50” and including an acrylic adhesive layer, is commercially available from Advanced Microdevices Pvt. Ltd., having a business address at 20-21 Industrial Area, Ambala Cantt 133 006, INDIA; www.mdimembrane.com. The entirety of the “Lateral Flow Test” section of the Advanced Microdevices Pvt. Ltd. product catalog, pages 2-5, including data sheet information for the L-H50 material, is hereby incorporated herein by reference.

The absorption pad 140 may be formed of a material having a structure suited to be highly absorbent, generating a wicking action facilitating the lateral flow of the blood plasma or serum across the migration membrane 102 to the test line 110. For example, the absorption pad 140 may be formed of a cellulosic fibrous material. Such a cellulosic material suitable for forming the absorption pad 140, having a nominal pore size of 3 μm, a thickness of about 330.2 μm, and a basis weight of about 186.3 g/m² is commercially available under the grade designation “BSP113PK Cellulose Absorbent 113” from the Pall Corporation. The entirety of the Pall Corporation's “Cellulose Absorbent Papers” 2-page product data sheet including information regarding BSP113PK Cellulose Absorbent 113 is hereby incorporated herein by reference.

The housing 142 may be formed of a material suited for fabricating a rigid protective container for the lateral-flow immuno-chromatographic assay device 100. For example, an organic polymeric material may be utilized.

In an example of a lateral-flow immuno-chromatographic assay device 100, the one or plurality of detection antibodies on the conjugate pad 104 may have specific binding affinity for a cardiac Troponin-I epitope. The plurality of detection antibodies may include cardiac Troponin-I antibody clone 19C7 together with either or both of cardiac Troponin-I antibody clones 4C2 and M155. Further, for example, the lateral-flow immuno-chromatographic assay device 100 may include one or a plurality of capture antibodies on the test line 110 having specific binding affinity for a cardiac Troponin-I epitope. The plurality of capture antibodies may include both cardiac Troponin-I antibody clone MF4 and cardiac Troponin-I antibody clone 16A11. The detection and capture antibodies may, as further examples, have specific binding affinity for cardiac Troponin-I in its free form, or fragmented forms, or phosphorylated forms, or in forms partially-digested by proteases, or as part of a complex with either or both of Troponin-T and Troponin-C, such as a cardiac Troponin-ITC complex. The detection and capture antibodies may have specific binding affinity for human cardiac Troponin-I epitopes. A threshold of sensitivity for a lateral-flow immuno-chromatographic assay device 100 utilized for qualitatively detecting cardiac Troponin-I in an assay sample including whole blood may be, for example, about 0.001 microgram/milliliter (μg/ml) equivalent to about 1.0 nanogram per milliliter (ng/ml), of cardiac Troponin-I in free form, or fragmented forms, or phosphorylated forms, or in forms partially-digested by proteases, or as part of a complex with either or both of Troponin-T and Troponin-C, such as a cardiac Troponin-ITC complex. As another example, the lateral-flow immuno-chromatographic assay device 100 may include first and second detection antibodies on the conjugate pad 104, wherein the lateral-flow immuno-chromatographic assay device 100 may include third and fourth capture antibodies on the test line 110, and wherein each of the first, second, third and fourth antibodies has specific binding affinity for a substantially different cardiac Troponin-I epitope. As examples, the first antibody may be cardiac Troponin-I antibody clone 19C7, the second antibody may be selected from cardiac Troponin-I antibody clones 4C2 and M155, the third antibody may be cardiac Troponin-I antibody clone MF4, and the fourth antibody may be cardiac Troponin-I antibody clone 16A11.

Suitable cardiac Troponin-I antibody clones, including clones 19C7, 4C2, M155, MF4 and 16A11, are commercially available under the grade designation 4T21 from HyTest Ltd., having a business address at Intelligate, Joukahaisenkatu 6, 20520 Turku, Finland (www.hytest.fi); and from Abcam plc, having a business address at 332 Cambridge Science Park, Cambridge CB4 0WN, England (www.abcam.com). Further cardiac Troponin-I antibodies that may be utilized are disclosed in “Markers of Cardiovascular Diseases and Metabolic Syndrome—II Troponin-specific Antibodies,” (pp. 14-22, 2009), published by HyTest Ltd. and downloaded from http://www.hytest.fi/data_sheets/Markers%20of%20Cardiovascular%20Diseases%20and%20Metabolic%20Syndrome.pdf, the entirety of which hereby is incorporated herein by reference. The Troponin-I detection and capture antibodies as obtained may be further diluted by their buffers, such as phosphate buffered saline (PBS) with sodium azide. Skeletal Troponin-I in its free form, fragmented forms, or as part of a complex with either or both of Troponin-T and Troponin-C may be utilized as a negative control. The detection and capture antibodies selected for utilization in a cardiac Troponin-I assay may be screened to rule out cross-reactivity with skeletal Troponin-I, by carrying out trials utilizing assay samples including skeletal Troponin-I. Cardiac Troponin-I in its free form, fragmented forms, or as part of a complex with either or both of Troponin-T and Troponin-C may be included in assay samples utilized in trials carried out as positive controls to verify the sensitivity and specificity of binding activity of the selected cardiac Troponin-I detection and capture antibodies. Suitable human cardiac Troponin-ITC complex is commercially available under the grade designation “8T62” from HyTest Ltd., chosen by AACC cTnI Standardization Subcommittee for international reference material. Suitable human cardiac Troponin-I is commercially available under the grade designation “8T53” from HyTest Ltd. Troponin-I—free blood serum may be utilized as another negative control. Suitable Troponin-I—free blood serum, purified by immunoaffinity chromatography, is commercially available under the grade designation “8TFS” from HyTest Ltd.

An assay sample to be tested may be pre-treated with an anti-coagulant such as a heparin salt, a citrate such as sodium citrate, or an EDTA salt before loading onto the pre-filter 108, to prevent coagulation of the assay sample while a diagnostic assay is being carried out. For example, an anti-coagulant—coated pipette or tube may be utilized. Alternatively, the material from which the pre-filter 108 is formed may be treated with an anti-coagulant. The lateral-flow immuno-chromatographic assay device 100 may be utilized, as examples, to qualitatively detect cardiac Troponin-I at a concentration within a range of between about 1 ng/ml and at least about 2,000 ng/ml.

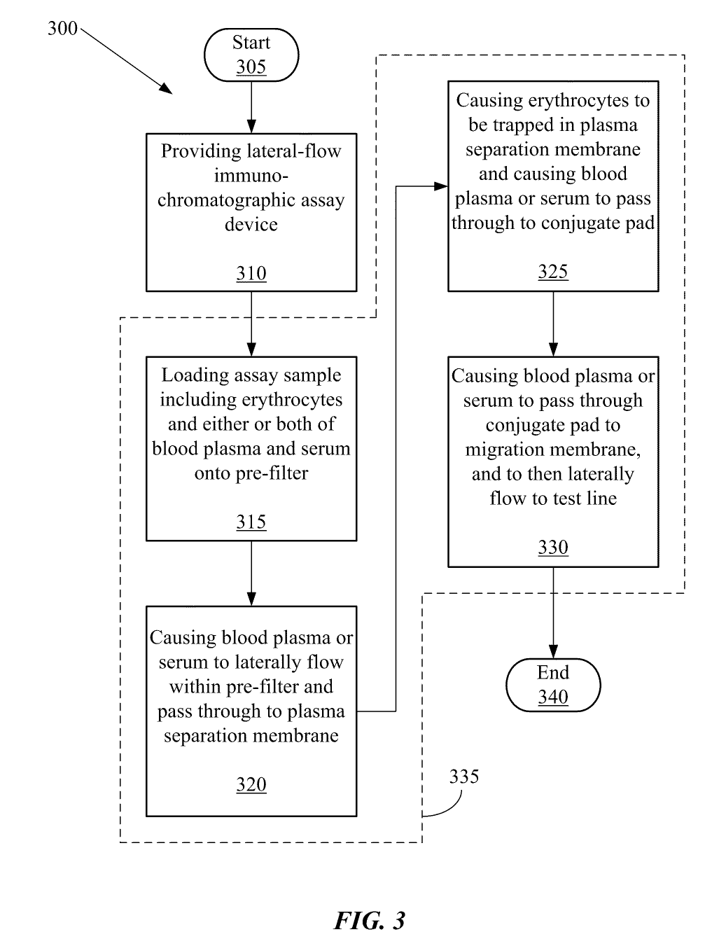

FIG. 3 is a flow diagram illustrating an example of an implementation of a method 300. The method 300 starts at step 305, and then step 310 includes providing a lateral-flow immuno-chromatographic assay device 100 including a migration membrane 102, a conjugate pad 104 being on the migration membrane 102, a plasma separation membrane 106 being on the conjugate pad 104, and a pre-filter 108 being on the plasma separation membrane 106; wherein the conjugate pad 104 is loaded with one or a plurality of detection antibodies having specific binding affinity for an assay target; and wherein the migration membrane 102 has a test line 110 loaded with one or a plurality of capture antibodies having specific binding affinity for the assay target. Step 310 may further include providing a lateral-flow immuno-chromatographic assay device 100 having any of the further features discussed earlier in connection with FIGS. 1-2. Step 315 includes loading an assay sample including erythrocytes and either or both of blood plasma and blood serum onto the pre-filter 108. The assay sample may include, in addition to erythrocytes, either blood plasma or blood serum alone, or blood plasma and blood serum together. Step 320 includes causing blood plasma or serum to laterally flow within the pre-filter 108 and allowing blood plasma or serum to pass through the pre-filter 108 to the plasma separation membrane 106. Step 325 includes causing erythrocytes to be trapped in the plasma separation membrane 106 and allowing blood plasma or serum to pass through the plasma separation membrane 106 and to flow to the conjugate pad 104. Step 330 includes allowing blood plasma or serum to pass through the conjugate pad 104 and to then flow onto the migration membrane 102 and to laterally flow across the migration membrane 102 to the test line 110. Steps 315, 320, 325 and 330 collectively define a diagnostic assay cycle 335. The method 300 may then end at step 340.

In an example, step 315 may include loading a chase buffer onto the pre-filter 108 after loading the assay sample onto the pre-filter 108, and step 330 may include causing the chase buffer to enhance lateral flow of blood plasma or serum across the migration membrane 102 to the test line 110. As an example, the chase buffer may include bovine serum albumin (BSA). Further, for example, a buffered saline solution including a nonionic detergent and a preservative such as sodium azide may be utilized as the chase buffer. As an example, the chase buffer may include, at a pH of 7.2: 10 mM 4-(2-hydroxyethyl)-1-piperazine ethanesulfonic acid (HEPES), 135 mM NaCl, 1% w/v BSA, and 50 milliliters per liter (mL/L) Tween 20. As another example, the chase buffer may include, at a pH of 7.8: 0.5% poly(ethylene glycol), 0.5% BSA, 0.1% Tween 20, and 0.1% MgCl₂ in Tris-buffered saline. As an additional example, the chase buffer may include 0.15M NaCl and 0.015M sodium citrate, supplemented with 1.4% Triton X-100 and 0.1% sodium dodecyl sulphate.

Step 320 may further include causing the blood plasma or serum to selectively pass through the pre-filter 108, and causing erythrocytes to be trapped on or in the pre-filter 108. Causing blood plasma or serum to laterally flow across the migration membrane 102 to the test line 110 at step 330 may include causing the leading edge of a visibly colored agent bound to the detection antibodies to be conveyed across the migration membrane 102 at a controlled speed, such as a controlled speed within a range of between about 2.5 min/3 cm and about 3.75 min/3 cm.

As an example, step 310 may include fabricating or obtaining a lateral-flow immuno-chromatographic assay device 100 that either has not yet been loaded with the detection antibodies or has not yet been loaded with the capture antibodies, or has not been loaded with the detection and capture antibodies, and then providing and loading such antibodies onto the lateral-flow immuno-chromatographic assay device 100. In another example, step 310 may include providing one or a plurality of detection antibodies having, and may include providing one or a plurality of capture antibodies having, specific binding affinity for a cardiac Troponin-I epitope. The plurality of detection antibodies may include cardiac Troponin-I antibody clone 19C7 together with either or both of cardiac Troponin-I antibody clones 4C2 and M155. The plurality of capture antibodies may include cardiac Troponin-I antibody clones MF4 and 16A11.

In another example, providing the lateral-flow immuno-chromatographic assay device 100 at step 310 may include loading first and second detection antibodies onto the conjugate pad 104 and loading third and fourth capture antibodies onto the test line 110; wherein each of the first, second, third and fourth antibodies has specific binding affinity for a substantially different cardiac Troponin-I epitope. For example, the first antibody may be cardiac Troponin-I antibody 19C7, the second antibody may be selected from cardiac Troponin-I antibody clones 4C2 and M155, the third antibody may be cardiac Troponin-I antibody clone MF4, and the fourth antibody may be cardiac Troponin-I antibody clone 16A11.

Step 315 of the method 300 may include utilizing an assay sample that includes whole blood. The assay sample may be a single drop of whole blood, such as a hanging drop. The method 300 may include collecting the whole blood from a human patient suspected of recently having suffered from or suspected of currently suffering from a myocardial infarction, also referred to as a heart attack.

In another example, the method 300 may include collecting another assay sample including whole blood or otherwise including erythrocytes together with either or both of blood plasma and serum from the same human patient, repeating step 310 to provide another lateral-flow immuno-chromatographic assay device 100, and carrying out another diagnostic assay cycle 335 utilizing the other assay sample and the other lateral-flow immuno-chromatographic assay device 100.

### EXAMPLES

In each of the following Examples, a lateral-flow immuno-chromatographic assay device 100 was fabricated. Each such device 100 included a substrate 138, a migration membrane 102 on the substrate 138, a conjugate pad 104 on the migration membrane 102, a plasma separation membrane 106 on the conjugate pad 104, and a pre-filter 108 on the plasma separation membrane 106. The pre-filters 108 and conjugate pads 104 in each of the lateral-flow immuno-chromatographic assay devices 100 were formed of a material including borosilicate glass fibers with a polyvinyl alcohol binder, having the grade designation SMCON64, obtained from the Pall Corporation. The plasma separation membranes 106 in each of the devices 100 were formed of a polysulfone material having the trade name “Vivid™ Plasma Separation Membrane”, also obtained from the Pall Corporation. The migration membranes 102 in each of the devices 100 were formed of a polyester-backed nitrocellulose membrane material sold under trade name “Vivid™ 170 Lateral Flow Nitrocellulose Membrane”, obtained from the Pall Corporation. Each migration membrane 102 included a defined test line 110 loaded with 1.4 μl of a dispersion including equal parts of mouse-derived cardiac Troponin-I antibody clones MF4 and 16A11 at concentrations of 0.9 mg/ml each, in 20 millimolar (mM) sodium phosphate buffer containing 2.5% v/v (2.5 milliliters per 100 milliliters) isopropanol. The migration membrane 102 was impregnated with a membrane blocking buffer dispersion containing 0.01M Na₂HPO₄, 0.5% w/v (grams per 100 milliliters) BSA, 0.1% w/v Tween 20, 0.5% v/v polyvinylpyrrolidone, 0.06M sucrose, and 0.05M NaCl. The substrate 138 was formed of L-H50, obtained from Advanced Microdevices Pvt. Ltd. Each conjugate pad 104 was loaded with 48 μl of a dispersion including equal parts of 40 nm colloidal gold-conjugated (at 10μg/ml) mouse-derived cardiac Troponin-I antibody clones 19C7 and 4C2 each at a concentration of 3 optical density units, in a dilution vehicle at pH 7.3 containing 5% w/v sucrose, 1.25% w/v trehalose, 0.01M disodium tetraborate decahydrate, 1% v/v polyvinyl acetate, 0.2% w/v Tween 20, and 0.2% w/v Triton-X 100. Each migration membrane 102 also included a defined internal control line 136 loaded with 1.4 μl of a dispersion including goat anti-mouse antibodies in sodium phosphate buffer, to verify proper assay functionality. Each of the devices 100 also included an absorption pad 140 formed of a cellulosic fibrous material. The above-identified components for each lateral-flow immuno-chromatographic assay device 100 were assembled together to form a structure as shown in FIGS. 1-2, and secured within a housing 142 also as shown in FIGS. 1-2. These components of the lateral-flow immuno-chromatographic assay devices 100 were held in position together by being moderately compressed between upper and lower halves of the housing 142.

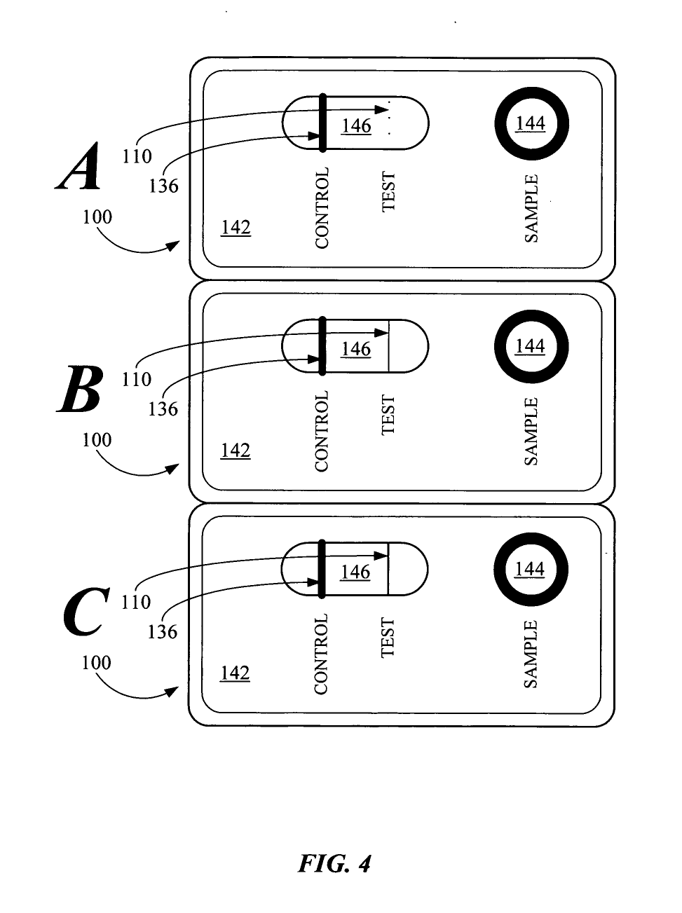

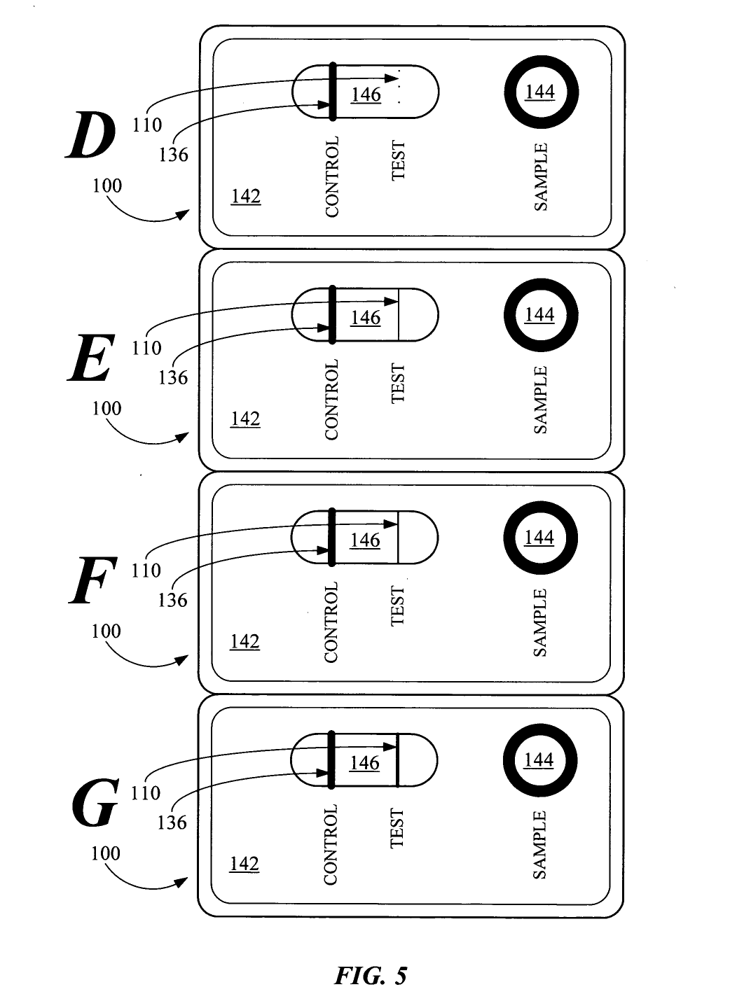

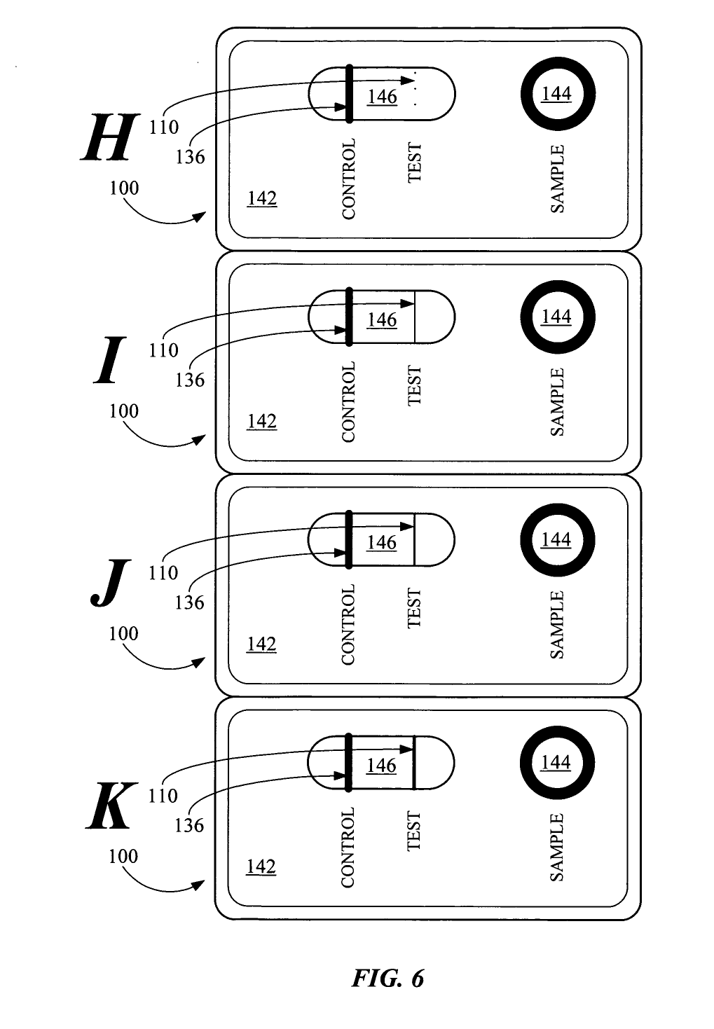

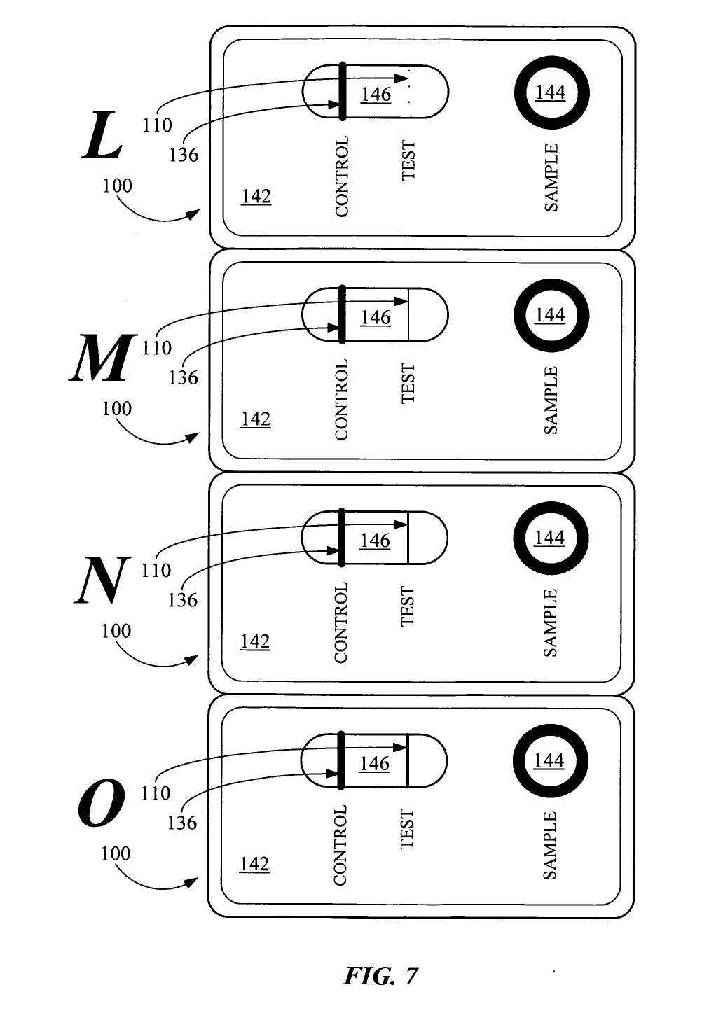

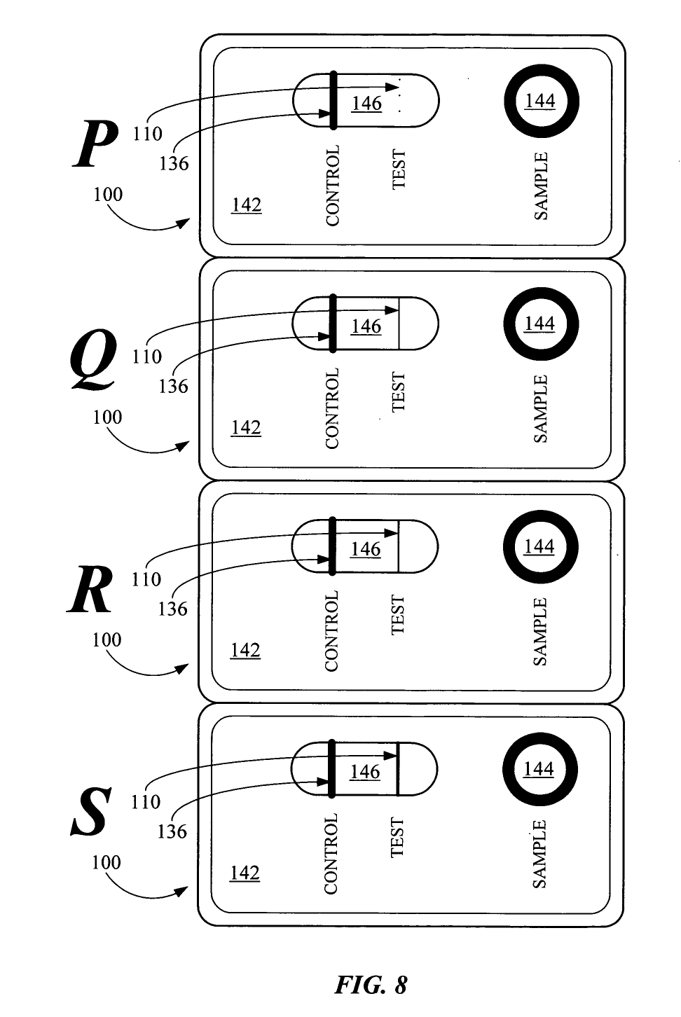

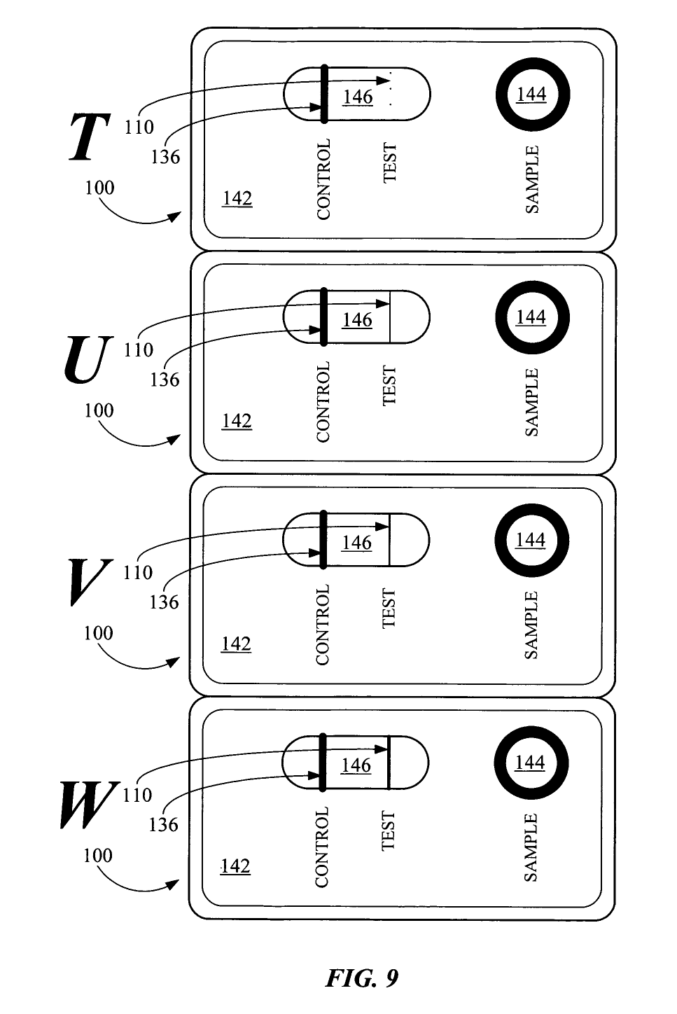

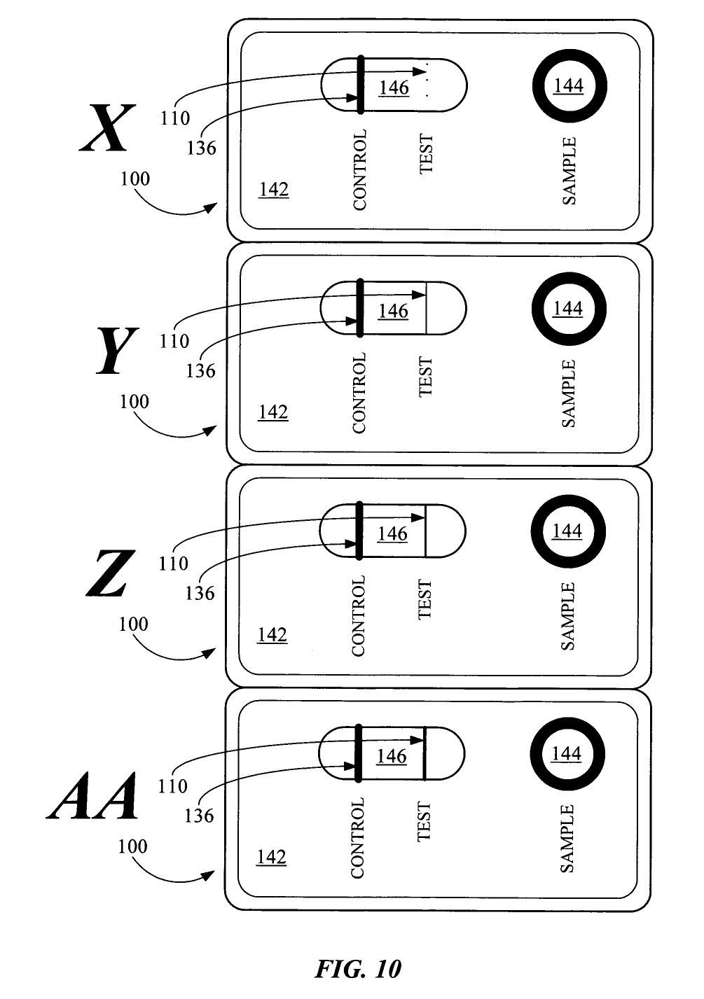

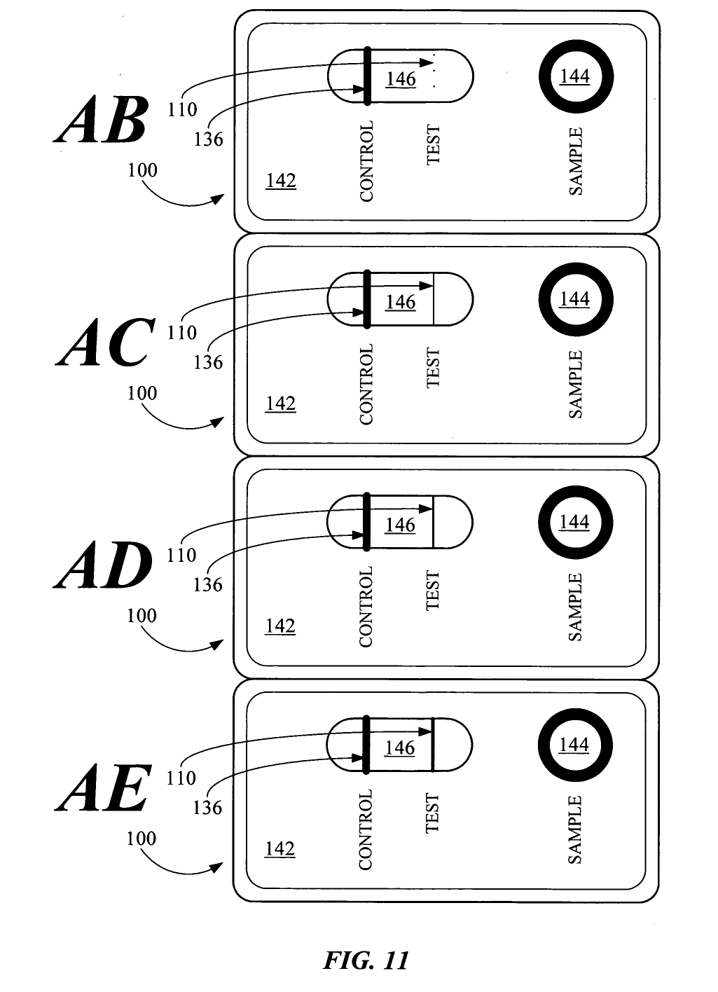

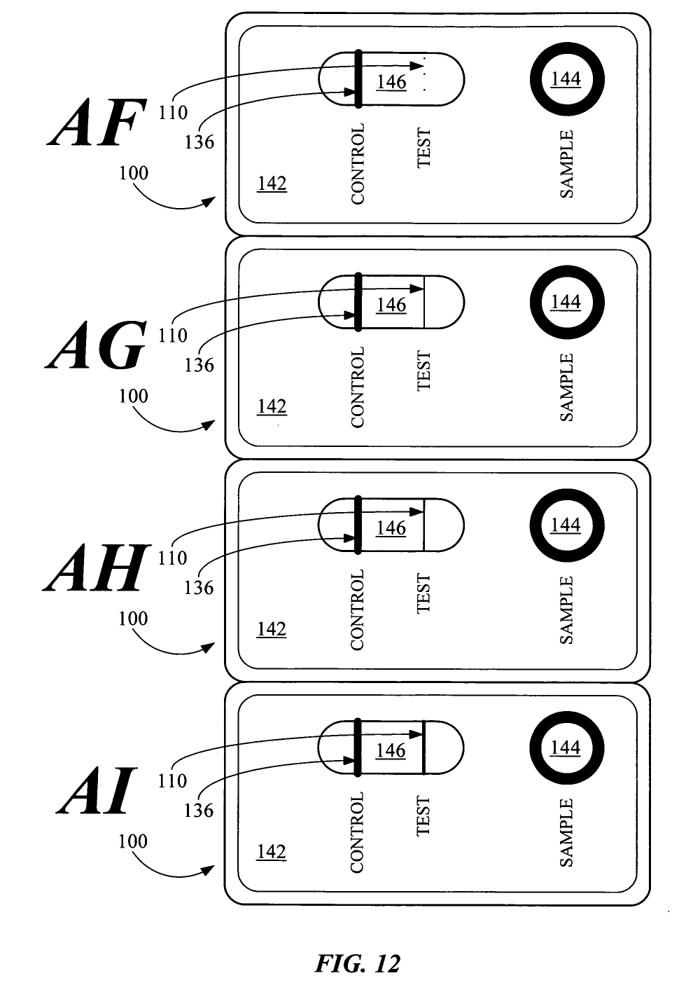

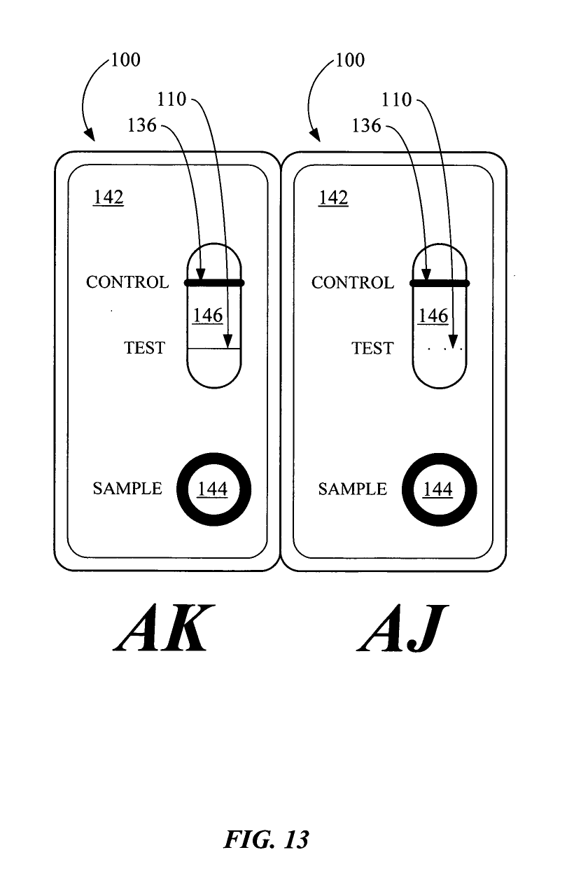

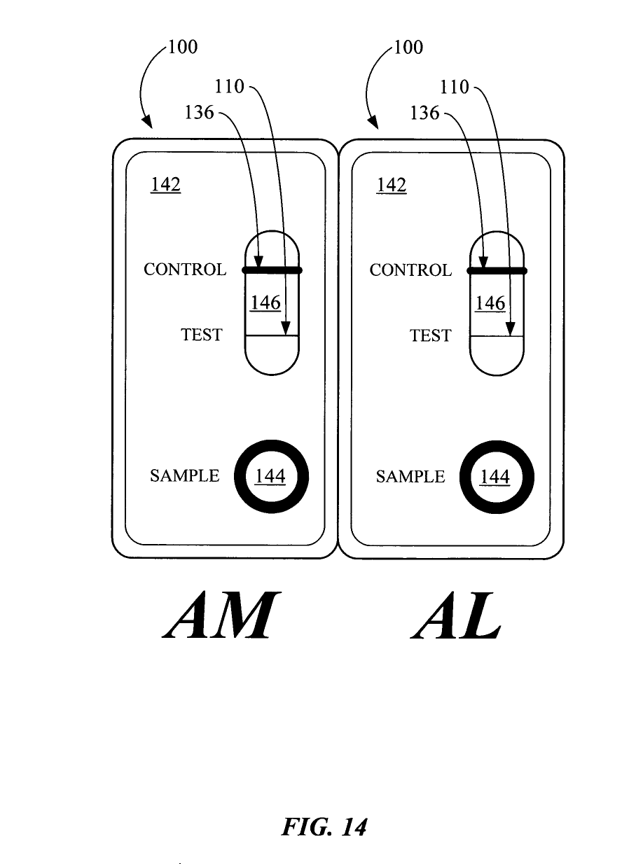

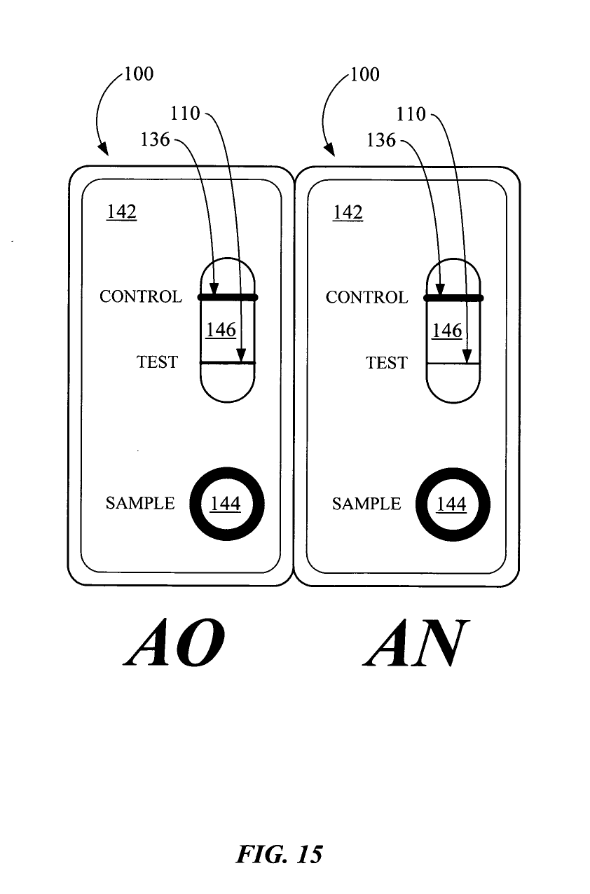

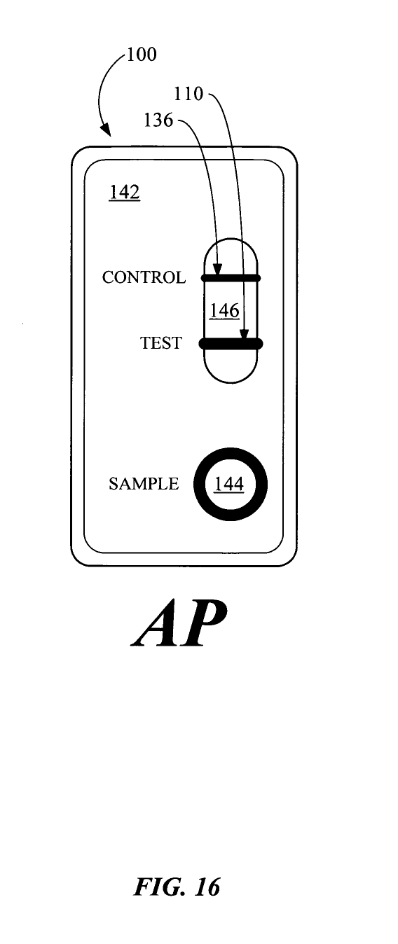

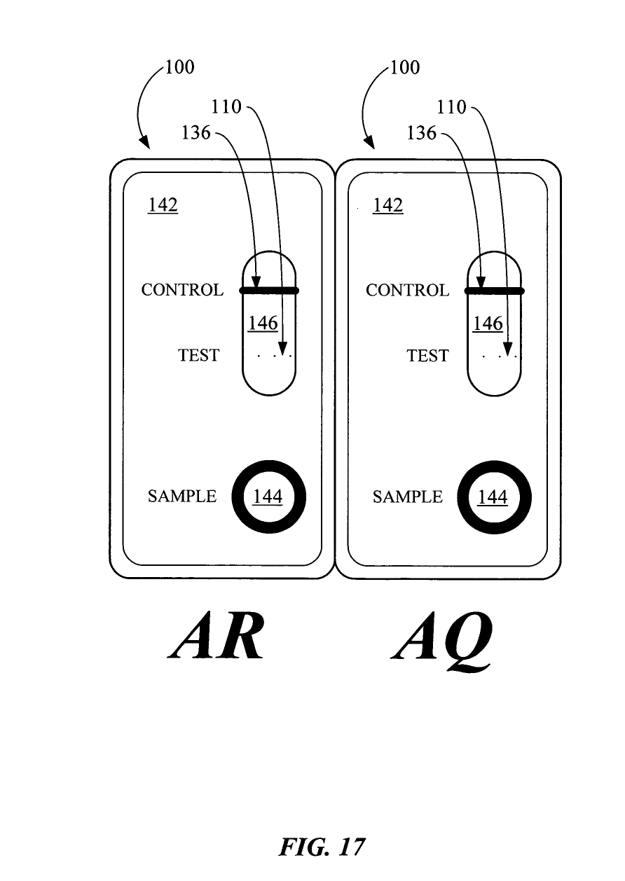

Each of the lateral-flow immuno-chromatographic assay devices 100 was then utilized to carry out a qualitative assay for the detection of human cardiac Troponin-ITC complex. Each assay sample was prepared by drawing a sample of whole blood having the volume of a hanging drop, supplemented with human cardiac Troponin-ITC complex at the concentrations in ng/ml indicated in Table 1, from a lithium-heparin-salt—containing tube. Each assay sample was loaded through an opening in the housing 142 onto the pre-filter 108, immediately followed by addition of 100 μl of a chase buffer. The human cardiac Troponin-ITC complex was obtained from HyTest Ltd., having the grade designation 8T62. Photographs of the lateral-flow immuno-chromatographic assay devices 100 showing their test lines 110 and control lines 136 were taken at the elapsed time periods indicated in Table 1 following the loading of the hanging drops of blood. The results are summarized in Table 1 and shown in FIGS. 4-17. In each photograph among FIGS. 4-17, the test lines 110 and control lines 136 of all of the lateral-flow immuno-chromatographic assay devices 100 shown in the photograph are mutually oriented in the same direction. The control lines 136 are to the left of the test lines 110 in Examples K, S, and AI; and the control line 136 is above the test line 110 in Example AK. Vivid control lines as shown in FIGS. 4-17 confirmed that the detection antibodies were carried to the test lines 110 in each of the examples.

Example B, using an assay sample having the volume of a hanging drop of whole blood including human cardiac Troponin-ITC complex at a concentration of 1 ng/ml, showed a faint positive test line after an elapsed test time period of about 15 minutes. Example C, using an assay sample having the volume of a hanging drop of whole blood including human cardiac Troponin-ITC complex at a concentration of 5 ng/ml, showed a clearly visible positive test line after the same elapsed test time period. Example A, serving as a negative control and carried out over the same elapsed test time period using an assay sample having the volume of a hanging drop of human whole blood and not supplemented with any added cardiac Troponin-ITC complex, did not yield a falsely-positive test line.

Example E, using an assay sample having the volume of a hanging drop of whole blood including human cardiac Troponin-ITC complex at a concentration of 1 ng/ml, showed a faint positive test line after an elapsed test time period of about 10 minutes. Examples I, M, Q, U, Y, AC and AG show that the faint positive test line remained visible after total elapsed test time periods of 11, 12, 15, 20, 25, 35 and 70 minutes, respectively. Each of Examples F, J, N, R, V, Z, AD and AH was carried out using an assay sample having the volume of a hanging drop of whole blood including human cardiac Troponin-ITC complex at a concentration of 5 ng/ml, yielding clearly visible positive test lines. Each of Examples G, K, O, S, W, AA, AE and AI was carried out using an assay sample having the volume of a hanging drop of whole blood including human cardiac Troponin-ITC complex at a concentration of 25 ng/ml, yielding vivid positive test lines. None of Examples D, H, L, P, T, X, AB or AF, each serving as a negative control and carried out using an assay sample having the volume of a hanging drop of human whole blood and not supplemented with any added cardiac Troponin-ITC complex, yielded any falsely-positive test lines.

Examples AJ through AO were carried out utilizing human whole blood having a concentration of hemolyzed blood cells sufficient to yield blood plasma having a visibly reddish color. Example AJ, serving as a negative control and carried out using an assay sample having the volume of a hanging drop of human whole blood and not supplemented with any added cardiac Troponin-ITC complex, did not yield a falsely-positive test line after an elapsed test time period of about 15 minutes. Examples AK and AL, each using an assay sample having the volume of a hanging drop of whole blood including human cardiac Troponin-ITC complex at a concentration of 1 ng/ml serving as a positive control, showed a faint positive test line after an elapsed test time period of about 15 minutes. Examples AM and AN, each using an assay sample having the volume of a hanging drop of whole blood including human cardiac Troponin-ITC complex at a concentration of 5 ng/ml, showed a clearly visible positive test line after an elapsed test time period of about 15 minutes. Example AO, using an assay sample having the volume of a hanging drop of whole blood including human cardiac Troponin-ITC complex at a concentration of 25 ng/ml, showed a vivid positive test line after an elapsed test time period of about 15 minutes.

Example AP, using an assay sample having the volume of a hanging drop of whole blood including human cardiac Troponin-ITC complex at a concentration of 1,000 ng/ml (equivalent to 1 μg/ml), showed a very vivid positive test line after an elapsed test time period of about 2 minutes.

In each of Examples AQ and AR, an assay sample was prepared by drawing a sample of whole blood, having the volume of a hanging drop supplemented with human skeletal Troponin-I instead of human cardiac Troponin ITC complex. The concentrations of human skeletal Troponin-I utilized in Examples AQ and AR were 25 ng/ml and 1,000 ng/ml, respectively. Neither of Examples AQ and AR yielded a falsely-positive test line after an elapsed test time period of about 15 minutes.

TABLE 1

| | FIG. | ITC | Elapsed | |
|---|---|---|---|---|
| Example | No. | ng/ml | Minutes | Qualitative Assay Result |
| A | 4 | 0 | 15 | Negative result at test line |
| B | 4 | 1 | 15 | Faint positive test line |
| C | 4 | 5 | 15 | Clearly visible positive test line |
| D | 5 | 0 | 10 | Negative result at test line |
| E | 5 | 1 | 10 | Faint positive test line |
| F | 5 | 5 | 10 | Clearly visible positive test line |
| G | 5 | 25 | 10 | Vivid positive test line |
| H | 6 | 0 | 11 | Negative result at test line |
| I | 6 | 1 | 11 | Faint positive test line |
| J | 6 | 5 | 11 | Clearly visible positive test line |
| K | 6 | 25 | 11 | Vivid positive test line |
| L | 7 | 0 | 12 | Negative result at test line |
| M | 7 | 1 | 12 | Faint positive test line |
| N | 7 | 5 | 12 | Clearly visible positive test line |
| O | 7 | 25 | 12 | Vivid positive test line |
| P | 8 | 0 | 15 | Negative result at test line |
| Q | 8 | 1 | 15 | Faint positive test line |
| R | 8 | 5 | 15 | Clearly visible positive test line |
| S | 8 | 25 | 15 | Vivid positive test line |
| T | 9 | 0 | 20 | Negative result at test line |
| U | 9 | 1 | 20 | Faint positive test line |
| V | 9 | 5 | 20 | Clearly visible positive test line |
| W | 9 | 25 | 20 | Vivid positive test line |
| X | 10 | 0 | 25 | Negative result at test line |
| Y | 10 | 1 | 25 | Faint positive test line |
| Z | 10 | 5 | 25 | Clearly visible positive test line |
| AA | 10 | 25 | 25 | Vivid positive test line |
| AB | 11 | 0 | 35 | Negative result at test line |
| AC | 11 | 1 | 35 | Faint positive test line |
| AD | 11 | 5 | 35 | Clearly visible positive test line |
| AE | 11 | 25 | 35 | Vivid positive test line |
| AF | 12 | 0 | 70 | Negative result at test line |
| AG | 12 | 1 | 70 | Faint positive test line |
| AH | 12 | 5 | 70 | Clearly visible positive test line |
| AI | 12 | 25 | 70 | Vivid positive test line |
| AJ | 13 | 0 | 15 | Negative result at test line |
| AK | 13 | 1 | 15 | Faint positive test line |
| AL | 14 | 1 | 15 | Faint positive test line |
| AM | 14 | 5 | 15 | Clearly visible positive test line |
| AN | 15 | 5 | 15 | Clearly visible positive test line |
| AO | 15 | 25 | 15 | Vivid positive test line |
| AP | 16 | 1,000 | 2 | Very vivid positive test line |
| AQ | 17 | 0 | 15 | Negative result at test line |
| AR | 17 | 0 | 15 | Negative result at test line |

It is understood that the method 300 may include fabricating, obtaining, or otherwise providing a lateral-flow immuno-chromatographic assay device 100 having any of the features included in the examples of lateral-flow immuno-chromatographic assay devices 100 discussed in this specification. It is further understood that the discussion herein of the lateral-flow immuno-chromatographic assay devices 100 illustrates suitable variations of the method 300. Likewise, it is understood that the discussion of the method 300 herein illustrates suitable variations of the lateral-flow immuno-chromatographic assay devices 100. Accordingly, the entire discussion of the lateral-flow immuno-chromatographic assay devices 100 is deemed incorporated into the discussion of the method 300. In addition, the entire discussion of the method 300 is deemed incorporated into the discussion of the lateral-flow immuno-chromatographic assay devices 100.

The lateral-flow immuno-chromatographic assay devices 100 may be utilized in carrying out qualitative sandwich immunoassays for detection of target antigens that may be found in an assay sample including whole blood, or otherwise including erythrocytes and either or both of blood plasma and blood serum; or an assay sample including either or both of blood plasma and blood serum and not including erythrocytes. Examples of such target antigens include proteins, viruses, bacteria, microbes, drugs of abuse, and other normal and abnormal constituents of human and non-human blood, blood plasma or serum.

While the foregoing description refers in some instances to the lateral-flow immuno-chromatographic assay devices 100, it is appreciated that the subject matter is not limited to these devices, or to the devices discussed in the specification. Devices having other configurations consistent with the foregoing teachings may be fabricated. Further, it is understood that the method 300 may include additional steps and modifications of the indicated steps. It will be understood that the foregoing description of numerous examples has been presented for purposes of illustration and description. This description is not exhaustive and does not limit the claimed invention to the precise forms disclosed. Modifications and variations are possible in light of the above description or may be acquired from practicing the invention. The claims and their equivalents define the scope of the invention.

We claim:

1. A device, comprising:
a migration membrane, having a test line configured for loading onto the test line of one or a plurality of capture antibodies having specific binding affinity for an assay target, the migration membrane being configured for allowing lateral flow of blood plasma or serum across the migration membrane to the test line;
a conjugate pad on the migration membrane, being configured for loading onto the conjugate pad of one or a plurality of detection antibodies having specific binding affinity for an assay target;
a plasma separation membrane on the conjugate pad, the plasma separation membrane being configured for allowing passage of blood plasma or serum through the plasma separation membrane and for trapping erythrocytes; and
a pre-filter on the plasma separation membrane, the pre-filter being configured for loading of an assay sample including erythrocytes and either or both of blood plasma and blood serum onto the pre-filter, the pre-filter being configured for allowing passage of blood plasma or serum through the pre-filter and configured for causing lateral flow of blood plasma or serum within the pre-filter.

2. The device of claim 1, wherein the plasma separation membrane has a first surface facing toward the pre-filter and a second surface facing toward the conjugate pad, wherein the plasma separation membrane includes a plurality of passageways each communicating with both of the first and second surfaces, and wherein a plurality of the passageways each has a first opening at the first surface and a second opening at the second surface, the second opening being smaller than the first opening.

3. The device of claim 2, wherein each of a plurality of the passageways has a frustoconical shape, the plurality of passageways being laterally spaced apart from each other within the plasma separation membrane.

4. The device of claim 3, wherein the frustoconical shape of each of the plurality of passageways is configured for trapping and immobilizing erythrocytes.

5. The device of claim 1, wherein the pre-filter has a random structure configured for allowing omnidirectional passage of blood plasma or serum through the pre-filter.

6. The device of claim 1, wherein the pre-filter has a random fibrous structure.

7. The device of claim 1, wherein the pre-filter is configured for causing selective passage of blood plasma or serum through the pre-filter and for trapping erythrocytes.

8. The device of claim 7, wherein the pre-filter is configured for trapping at least about 10% of a quantity of erythrocytes from an assay sample.

9. The device of claim 1, wherein the pre-filter has an exposed first surface and a second surface facing toward the plasma separation membrane, wherein the pre-filter has an asymmetric structure including a plurality of passageways each having a first opening communicating with the first surface and a second opening communicating with the second surface, and wherein an average spacing between the second openings is larger than an average spacing between the first openings.

10. The device of claim 1, wherein the pre-filter and the plasma separation membrane are collectively configured for trapping at least about 90% of a quantity of erythrocytes from an assay sample.

11. The device of claim 1, wherein the device is configured for conveying between about 60% by volume and about 80% by volume of the blood plasma or serum from an assay sample to the migration membrane.

12. The device of claim 11, including configuring the device as capable of utilizing a single drop of whole blood as the assay sample.

13. The device of claim 1, wherein the migration membrane has a longitudinal axis, wherein the plasma separation membrane has a midpoint tangentially located over a first point along the longitudinal axis, wherein the conjugate pad has a midpoint tangentially located over a second point along the longitudinal axis, and wherein the second point is nearer to the test line than is the first point.

14. The device of claim 1, wherein the detection antibodies include a bound visibly colored agent, and wherein the migration membrane is configured for causing a leading edge of the visibly colored agent to be conveyed across the migration membrane at a controlled speed within a range of between about 2.5 minutes per 3 centimeters and about 3.75 minutes per 3 centimeters.

15. The device of claim 14, wherein the migration membrane is impregnated with a membrane blocking buffer at a concentration selected for causing blood plasma or serum to be conveyed at the controlled speed.

16. The device of claim 14, wherein the migration membrane has an average pore diameter selected for causing blood plasma or serum to be conveyed at the controlled speed.

17. The device of claim 14, wherein the migration membrane is electrically uncharged.

18. The device of claim 1, wherein the one detection antibody has or the plurality of detection antibodies have specific binding affinity for a cardiac Troponin-I epitope.

19. The device of claim 18, wherein the plurality of detection antibodies includes cardiac Troponin-I antibody clone 19C7 together with either or both of cardiac Troponin-I antibody clones 4C2 and M155.

20. The device of claim 1, wherein the device includes one or a plurality of capture antibodies on the test line having specific binding affinity for a cardiac Troponin-I epitope.

21. The device of claim 20, wherein the plurality of capture antibodies includes cardiac Troponin-I antibody clone MF4 and cardiac Troponin-I antibody clone 16A11.

22. The device of claim 1, wherein the device includes first and second detection antibodies on the conjugate pad, wherein the device includes third and fourth capture antibodies on the test line, and wherein each of the first, second, third and fourth antibodies has specific binding affinity for a substantially different cardiac Troponin-I epitope.

23. The device of claim 22, wherein the first antibody is cardiac Troponin-I antibody 19C7, the second antibody is selected from cardiac Troponin-I antibody clones 4C2 and M155, the third antibody is cardiac Troponin-I antibody clone MF4, and the fourth antibody is cardiac Troponin-I antibody clone 16A11.

24. A method, comprising:
providing a lateral-flow immuno-chromatographic assay device including a migration membrane, a conjugate pad being on the migration membrane, a plasma separation membrane being on the conjugate pad, and a pre-filter being on the plasma separation membrane; wherein the conjugate pad is loaded with one or a plurality of detection antibodies having specific binding affinity for an assay target; and wherein the migration membrane has a test line loaded with one or a plurality of capture antibodies having specific binding affinity for the assay target; and carrying out a diagnostic assay cycle, including:
loading an assay sample including erythrocytes and either or both of blood plasma and blood serum onto the pre-filter;
causing blood plasma or serum to laterally flow within the pre-filter and allowing blood plasma or serum to pass through the pre-filter to the plasma separation membrane;
causing erythrocytes to be trapped in the plasma separation membrane and allowing blood plasma or serum to pass through the plasma separation membrane to the conjugate pad; and
allowing blood plasma or serum to pass through the conjugate pad onto the migration membrane and allowing blood plasma or serum to laterally flow across the migration membrane to the test line.

25. The method of claim 24, including causing erythrocytes to be trapped on the pre-filter and causing blood plasma or serum to selectively pass through the pre-filter.

26. The method of claim 24, including loading a chase buffer onto the pre-filter after loading the assay sample onto the pre-filter, and causing the chase buffer to enhance lateral flow of blood plasma or serum across the migration membrane to the test line.

27. The method of claim 24, wherein the detection antibodies include a bound visibly colored agent, and wherein allowing blood plasma or serum to laterally flow across the migration membrane to the test line includes causing a leading edge of the visibly colored agent to be conveyed across the migration membrane at a controlled speed within a range of between about 2.5 minutes per 3 centimeters and about 3.75 minutes per 3 centimeters.

28. The method of claim 24, wherein the one or plurality of detection antibodies have, and wherein the one or plurality of capture antibodies have, specific binding affinity for a cardiac Troponin-I epitope.

29. The method of claim 28, wherein the plurality of detection antibodies includes cardiac Troponin-I antibody clone 19C7 together with either or both of cardiac Troponin-I antibody clones 4C2 and M155.

30. The method of claim 28, wherein the plurality of capture antibodies includes cardiac Troponin-I antibody clones MF4 and 16A11.

31. The method of claim 27, wherein providing the lateral-flow immuno-chromatographic assay device includes loading first and second detection antibodies onto the conjugate pad and loading third and fourth capture antibodies onto the test line; and wherein each of the first, second, third and fourth antibodies has specific binding affinity for a substantially different cardiac Troponin-I epitope.

32. The method of claim 31, wherein the first antibody is cardiac Troponin-I antibody clone 19C7, the second antibody is selected from cardiac Troponin-I antibody clones 4C2 and M155, the third antibody is cardiac Troponin-I antibody clone MF4, and the fourth antibody is cardiac Troponin-I antibody clone 16A11.

33. The method of claim 24, wherein the assay sample includes whole blood.

34. The method of claim 33, wherein the assay sample is a single drop of whole blood.

35. The method of claim 33, wherein the method includes collecting the whole blood from a human patient suspected of recently having suffered from or suspected of currently suffering from a myocardial infarction.

36. The method of claim 35, including collecting another assay sample including whole blood from the human patient, providing another lateral-flow immuno-chromatographic assay device, and utilizing the other assay sample and the other lateral-flow immuno-chromatographic assay device for carrying out another diagnostic assay cycle.
